(19) United States
(12) Patent Application Publication
(10) Pub. No.: US 2025/0250316 A1
(43) Pub. Date: Aug. 7, 2025

(54) DESIGN AND COMPOSITION OF HUANTI-CD19 CHIMERIC ANTIGEN RECEPTOR TARGETING B-CELL MALIGNANCIES THEREOF

(71) Applicant: LYSINE BIOTECH PRIVATE LIMITED, Tamil Nadu (IN)

(72) Inventor: Ananda Gopu PERUMAL, Tamil Nadu (IN)

(21) Appl. No.: 18/897,292

(22) Filed: Sep. 26, 2024

(30) Foreign Application Priority Data
Sep. 27, 2023 (IN) 202341064804

(51) Int. Cl. C07K 14/725; C07K 16/28

(52) U.S. Cl. CPC C07K 14/7051; C07K 16/2803; C07K 2317/24; C07K 2317/622

(57) ABSTRACT

The present invention provides to develop novel chimeric antigen receptor (CAR) encoded by an open reading frame 3 (ORF3) mRNA and amino acid sequences of any one of SEQ ID NO: 1 to SEQ ID NO: 109 specific to CD19 against hematologic malignancies associated with expression of Cluster of Differentiation 19 (CD19). The invention relates to the design of a synthetic CAR mRNA sequence comprising hu anti CD19 scFv, a hinge, a Transmembrane domain, a co-stimulatory domain and a CD3ζ signaling domain, where with the costimulatory is CD27 or 41BB for targeting and destroying malignant B-cells. This disclosure features anti-CD19 CAR T-cell therapy for antigen binding domains, directed to B cell malignancies, described herein.

### FIELD OF INVENTION

### Cross-Reference to Related Application

This application claims the benefit or priority of Indian patent application no. 202341064804 filed Sep. 27, 2023, the contents of which is incorporated by reference in its entirety.

### SEQUENCE LISTING

The instant application contains a Sequence Listing XML, which has been submitted electronically in XML format and is hereby incorporated by reference in its entirety. Said XML copy, created on Oct. 2, 2024, is named 130345.021500_Sequence_listing.xml and is 164,459 bytes in size.

### FIELD

The present disclosure is related to development of synthetic chimeric antigen receptor (CAR) against CD19. Particularly, the present disclosure relates to designing of synthetic human anti-CD19 CAR which will be expressed by T cells. Particularly, the present disclosure relates to anti-CD19 CAR T cell therapy for B cell malignancies.

### BACKGROUND OF INVENTION

Many patients with B-cell malignancies are incurable with conventional therapy, however traditional treatments often associate with serious side effects. Recent developments in cancer immunotherapy has overcome several challenges and have shown promising clinical outcomes. Genetically modified T cells expressing chimeric antigen receptors (CAR) administered in patients are designed to target specific antigen expressing malignant B cells such as CD19, and reported effective complete remission in patients with B-cell malignancies. CAR is engineered majorly with single chain variable fragment (scFv) linked with a hinge and transmembrane domain to one or more intracellular signaling domains. T cells isolated from patients will be modified to express CAR on their surface and redirected to target the antigen expressing tumor cells in hematologic and solid tumors.

Currently, there are 6 FDA approved CART therapies for the treatment of young and adults relapse/refractory B-ALL. Patients undergoing personalized CART cell therapy have shown improved remission and survival rate. However, existing CART therapies are associated with major drawbacks such as cytokine release syndrome, B cell aplasia, neurotoxicity etc. Hence, it's important to carefully design the CAR construct to minimize toxic side effects and generate CART cells with longer persistence and reduced cytotoxicity. Hence, there's a need for the development of specific antigen targeted CARs for the treatment of tumor malignancies with reduced toxicities, improved efficacy, higher stability and cost effective manufacturability.

### SUMMARY OF INVENTION

In an embodiment, the invention provides a chimeric antigen receptor comprising (a) a promoter (b) leader sequence, (c) single chain variable fragment (scFv) as an antigen-specific targeting region, (d) a hinge region representing extracellular spacer domain, (e) a transmembrane domain, (f) at least one co-stimulatory signaling domain and (g) an intracellular signaling domain. In an embodiment, the invention provides an antigen specific targeting region comprises a hu CD-19 specific single chain Fragment variable (scFv) fragment, that binds to the CD19 expressing malignant B cells.

The present invention in specific embodiments relates to nucleic acid encoding huCAR, wherein huCAR comprises:
a) a signal sequence/leader sequence;
b) a scFv or fragment comprising a hu anti-CD19 binding domain as an antigen specific targeting region;
c) a hinge region;
d) a transmembrane domain;
e) a cytoplasmic domain comprising at least one co-stimulatory domain;
f) one intracellular signaling domain; and
g) optionally a promoter sequence selected from EF-1α or MND, and
wherein the scFv or fragment comprising a hu anti-CD19 binding domain, comprises a heavy chain variable region, a light chain variable region; and wherein the heavy chain variable region comprises a sequence of SEQ ID. NO.: 1, and wherein the light chain variable region comprises a sequence of SEQ ID. NO.:2.

The present invention in specific embodiments also relates to a nucleic acid encoding huCAR,
wherein huCAR comprises;
a) a signal sequence/leader sequence;
b) a scFv or fragment comprising a hu anti-CD19 binding domain as an antigen specific targeting region;
c) a hinge region;
d) a transmembrane domain;
e) a cytoplasmic domain comprising at least one co-stimulatory domain;
f) one intracellular signaling domain; and
g) optionally a promoter sequence selected from EF-1α or MND, and
wherein scFv or fragment comprising a hu anti-CD19 binding domain, comprises a heavy chain variable region, a light chain variable region; and
wherein the costimulatory domain is selected from amino acid sequences of SEQ ID NO: 6, SEQ ID NO: 9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, or SEQ ID NO:34, or a sequence with 95-99% identity to an amino acid sequence of SEQ ID NO: 6, SEQ ID NO: 9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, or SEQ ID NO:34.

In specific embodiments the invention relates to nucleic acid encoding huCAR, wherein huCAR comprises;
a) a signal sequence/leader sequence;
b) a scFv or fragment comprising a hu anti-CD19 binding domain as an antigen specific targeting region;
c) a hinge region;
d) a transmembrane domain;
e) a cytoplasmic domain comprising at least one co-stimulatory domain;
f) one intracellular signaling domain; and
g) optionally a promoter sequence selected from EF-1α or MND, and
wherein scFv or fragment comprising a hu anti-CD19 binding domain, comprises a heavy chain variable region, a light chain variable region; and
wherein the costimulatory domain comprises a sequence selected from SEQ ID NO: 7, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, or SEQ ID NO:49 or a sequence with 95-99% identity to an amino acid sequence provided in SEQ ID NO.:7, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49.

The nucleic acid construct of the invention comprises a heavy chain variable region encoding framework, a light chain variable region encoding framework; and wherein the heavy chain encoding framework region comprises a sequence of SEQ ID. NO.: 102, and wherein the light chain encoding framework region comprises a sequence of SEQ ID. NO.: 103. The encoded heavy chain variable region (HCVR) and the light chain variable region (LCVR) comprises an amino acid sequence having at least one, two or three modifications but not more than 10/20 modifications of an amino acid sequence or a sequence with 95-99% identity to an amino acid sequence of SEQ ID NO 1 and SEQ ID NO: 2 thereof.

The hu anti-CD19 binding domain comprises a heavy chain variable region, a light chain variable region and wherein, the encoded heavy chain variable region is attached to the encoded light chain variable region via a linker. In specific embodiments of the invention, the linker comprises an amino acid sequence of SEQ ID NO: 3 and it is encoded by a sequence of SEQ ID. NO.: 104.

In specific embodiments of the invention, the nucleic acid construct of the invention encodes for a hu-CAR which is used for treating a subject having a disease associated with expression of a CD19.

The present invention is also directed to an huCAR, wherein huCAR comprises;
a. a signal sequence/leader sequence;
b. a scFv or fragment comprising a hu anti-CD19 binding domain as an antigen specific targeting region;
c. a hinge region;
d. a transmembrane domain;
e. a cytoplasmic domain comprising at least one co-stimulatory domain;
f. one intracellular signaling domain; and
g. optionally a promoter sequence selected from EF-1α or MND, and
wherein scFv or fragment comprising a hu anti-CD19 binding domain, comprises a heavy chain variable region, a light chain variable region; and wherein heavy chain variable region comprises a sequence of SEQ ID. NO.: 1, and wherein the light chain variable region comprises a sequence of SEQ ID. NO.: 2.

The present invention is also directed to an huCAR, wherein the huCAR comprises;
a. a signal sequence/leader sequence;
b. a scFv or fragment comprising a hu anti-CD19 binding domain as an antigen specific targeting region;
c. a hinge region;
d. a transmembrane domain;
e. a cytoplasmic domain comprising at least one co-stimulatory domain;
f. one intracellular signaling domain; and
g. optionally a promoter sequence selected from EF-1α or MND, and
wherein scFv or fragment comprising a hu anti-CD19 binding domain, comprises a heavy chain variable region, a light chain variable region; and wherein the costimulatory domain is selected from amino acid sequences of SEQ ID NO: 6, SEQ ID NO: 9, SEQ ID NO:10, SEQ ID NO:11, SEQ ID NO:12, SEQ ID NO:13, SEQ ID NO:14, SEQ ID NO:15, SEQ ID NO:16, SEQ ID NO: 17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, or SEQ ID NO:34, or a sequence with 95-99% identity to an amino acid sequence of SEQ ID NO: 6, SEQ ID NO: 9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO: 18, SEQ ID NO: 19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, or SEQ ID NO: 34.

The present invention is also directed to an huCAR, wherein huCAR comprises;
a. a signal sequence/leader sequence;
b. a scFv or fragment comprising a hu anti-CD19 binding domain as an antigen specific targeting region;
c. a hinge region;
d. a transmembrane domain;
e. a cytoplasmic domain comprising at least one co-stimulatory domain;
f. one intracellular signaling domain; and
g. optionally a promoter sequence selected from EF-1α or MND, and
wherein scFv or fragment comprising a hu anti-CD19 binding domain, comprises a heavy chain variable region, a light chain variable region; and
wherein the costimulatory domain comprises a sequence selected from SEQ ID NO: 7, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, or SEQ ID NO:49 or a sequence with 95-99% identity to an amino acid sequence provided in SEQ ID NO.:7, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49 thereof.

In an embodiment, the anti CD-19 binding domain comprises a heavy chain from selected SEQ ID NO: 1 and encoded by sequence 102 and a light chain of SEQ ID NO: 2 & encoded by sequence 103.

In an embodiment, provides a peptide linker that connects the heavy chain and light chain of scFv and the encoded linker comprises an amino acid sequence of SEQ ID NO: 3 & enclosed by nucleic acid sequence of 104.

In an embodiment, the invention further provides a chimeric antigen receptor, comprising (a) a CD8a hinge extracellular spacer domain, (b) a CD8α transmembrane domain, (d) a cytoplasmic domain comprising a human CD27 and/or 4-1BB costimulatory domain and a CDζ (intracellular signaling domain.

In an embodiment, a hinge domain connects the scFv with the transmembrane domain providing flexibility to the scFv domain for recognition of the target epitope. The hinge domain maintaining the optimal synapse distance comprises an amino acid sequence of SEQ ID NO: 5 In an embodiment, the transmembrane domain is encoded by SEQ ID NO: 106 and comprises an amino acid sequence of SEQ ID NO: 5.

In an embodiment, the costimulatory domain comprises an amino acid sequence of SEQ ID NO: 6 & SEQ ID NO: 7 provides the signal for proliferation, metabolic cycles and activates transcription factors for the production of cytokines.

In an embodiment, recombinant nucleic acid sequence of hu anti-CD19 CAR comprising 4-1BB as costimulatory domain is selected from group of sequences of SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO:85, and SEQ ID NO: 86.

In an embodiment, recombinant nucleic acid sequence of hu anti-CD19 CAR comprising CD27 as costimulatory domain is selected from group of sequences of SEQ ID NO: 87, SEQ ID NO: 88, SEQ 30 ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, and SEQ ID NO: 101.

In an embodiment, the intracellular signaling domain comprises an amino acid sequence of SEQ ID NO: 8.

In an embodiment, the promoter sequence for EF-1α and MND comprises an amino acid sequence of SEQ ID NO. 50, SEQ ID NO. 51, SEQ ID NO. 52, SEQ ID NO. 53, SEQ ID NO. 54, SEQ ID NO. 55, SEQ ID NO. 56, SEQ ID NO. 57, SEQ ID NO. 58, SEQ ID NO. 59, and SEQ ID NO. 60

In an embodiment, the B-cell malignancies such as B-cell chronic lymphocytic leukemia, acute lymphoblastic leukemia, B-cell non-Hodgins lymphoma including other CD19 positive malignancies is selected for providing treatment using the present invention.

In a further aspect, this invention pertains to the nucleic acid molecule of the invention, nucleic acid molecule anti-CD19 CAR, cell comprising the nucleic acid molecules of the invention for the treatment of CD-19 associated diseases.

### BRIEF DESCRIPTION OF DRAWINGS

FIGS. 1A-1B provide a synthetic construct of hu anti-CD19-4-1BBZ (KCAR), FIG. 1C provides a schematic representation of the construct, and FIG. 1D provides its predictive signaling network.

FIGS. 2A-2B provide a synthetic construct of hu anti-CD19-CD27Z (LYSCAR).

FIG. 2C provides a schematic representation of the construct, and FIG. 2D provides its predictive signaling network.

FIGS. 3A-3B provide tables listing the Amino acid sequence of CDR regions in heavy chain and light chains in hu anti-CD19 CAR.

FIG. 4 provides a table listing the components of hu anti-CD19 CAR.

FIGS. 5A-5B provide tables listing the intracellular signaling domain of hu anti-CD19 CAR.

FIG. 6—Table listing the promoter sequence.

### DETAILED DESCRIPTION

All references cited herein are incorporated by reference in their entirety as though fully set forth.

Unless defined, all the technical and scientific terms used herein have the same meaning as commonly understood by one of ordinary skill in the art to which invention belongs. One skilled in the art will recognize many methods and materials similar or equivalent to those described herein, which could be used in the practice of the present invention. Indeed, the present invention is not limited to the methods and/or materials and/or process parameters as provided in the following detailed description.

The present invention, the term hu as used herein, refers to nucleic acid or amino acid sequence derived proteins isolated from human.

The present invention provides details on development of a hu chimeric antigen receptor (CAR) where the recombinant nucleic acid molecule comprises a hu anti-CD19 binding domain, that targets highly conserved CD19 expressing B cell tumors as in case of B-cell malignancies including leukemia and lymphomas.

A CAR is engineered by connecting the extracellular domain, with one or more intracellular domain connected via a transmembrane domain. Based on the antigen targeted on cancer cells, extracellular domain has the antigen binding domain derived from a monoclonal antibody. The intracellular domain of CAR is derived from T cell receptors in cytoplasmic domain and primes the CAR expressing T cells against target antigen on extracellular domain. A transmembrane domain connects intracellular domain to extracellular domain, which is either derived naturally or designed as per requirements.

As described herein, the present invention comprises a hu CD19 binding domain provides anti-tumor activity against CD-19 expressing tumor cells. According to the present invention, the hu anti-CD19 chimeric antigen receptor is a polypeptide sequence, that comprises:
(a) A Signal sequence/leader sequence
(b) a scFv comprising a hu anti-CD19 binding domain
(c) a hinge region
(d) a transmembrane domain
(e) a co-stimulatory domain for CD27 and/or 41BB
(f) a CDζ (intracellular signaling domain
(g) a promoter

The present invention in specific embodiments relates to nucleic acid encoding huCAR, wherein huCAR comprises:
a) a signal sequence/leader sequence;
b) a scFv or fragment comprising a hu anti-CD19 binding domain as an antigen specific targeting region;
c) a hinge region;
d) a transmembrane domain;
e) a cytoplasmic domain comprising at least one co-stimulatory domain;
f) one intracellular signaling domain; and
g) optionally a promoter sequence selected from EF-1α or MND, and
wherein the scFv or fragment comprising a hu anti-CD19 binding domain, comprises a heavy chain variable region, a light chain variable region; and wherein the heavy chain variable region comprises a sequence of SEQ ID. NO.: 1, and wherein the light chain variable region comprises a sequence of SEQ ID. NO.:2.

The present invention in specific embodiments also relates to a nucleic acid encoding huCAR,
wherein huCAR comprises;
a) a signal sequence/leader sequence;
b) a scFv or fragment comprising a hu anti-CD19 binding domain as an antigen specific targeting region;
c) a hinge region;
d) a transmembrane domain;
e) a cytoplasmic domain comprising at least one co-stimulatory domain;
f) one intracellular signaling domain; and
g) optionally a promoter sequence selected from EF-1α or MND, and
wherein scFv or fragment comprising a hu anti-CD19 binding domain, comprises a heavy chain variable region, a light chain variable region; and
wherein the costimulatory domain is selected from amino acid sequences of SEQ ID NO: 6, SEQ ID NO: 9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, or SEQ ID NO:34, or a sequence with 95-99% identity to an amino acid sequence of SEQ ID NO: 6, SEQ ID NO: 9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, or SEQ ID NO:34.

In specific embodiments the invention relates to nucleic acid encoding huCAR, wherein huCAR comprises;
a) a signal sequence/leader sequence;
b) a scFv or fragment comprising a hu anti-CD19 binding domain as an antigen specific targeting region;
c) a hinge region;
d) a transmembrane domain;
e) a cytoplasmic domain comprising at least one co-stimulatory domain;
f) one intracellular signaling domain; and
g) optionally a promoter sequence selected from EF-1α or MND, and
wherein scFv or fragment comprising a hu anti-CD19 binding domain, comprises a heavy chain variable region, a light chain variable region; and
wherein the costimulatory domain comprises a sequence selected from SEQ ID NO: 7, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, or SEQ ID NO:49 or a sequence with 95-99% identity to an amino acid sequence provided in SEQ ID NO.:7, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49.

The nucleic acid construct of the invention comprises a heavy chain variable region encoding framework, a light chain variable region encoding framework; and wherein the heavy chain encoding framework region comprises a sequence of SEQ ID. NO.: 102, and wherein the light chain encoding framework region comprises a sequence of SEQ ID. NO.: 103. The encoded heavy chain variable region (HCVR) and the light chain variable region (LCVR) comprises an amino acid sequence having at least one, two or three modifications but not more than 10/20 modifications of an amino acid sequence or a sequence with 95-99% identity to an amino acid sequence of SEQ ID NO 1 and SEQ ID NO: 2 thereof.

The nucleic acid construct of the invention comprises a region that enclosed for a scFv or fragment comprising a hu anti-CD19 binding domain. The hu anti-CD19 binding domain comprises a heavy chain variable region, a light chain variable region and wherein, the encoded heavy chain variable region is attached to the encoded light chain variable region via a linker.

In specific embodiments of the invention, the nucleic acid construct of the invention encodes for a hu-CAR that comprises a linker and the linker comprises an amino acid sequence of SEQ ID NO: 3 and it is encoded by a sequence of SEQ ID. NO.: 104.

In specific embodiments of the invention, the nucleic acid construct of the invention encodes for a hu-CAR that comprises a transmembrane domain which is a protein derived from a human CD8α.

In specific embodiments of the invention, the nucleic acid construct of the invention encodes for a hu-CAR that comprises an anti-CD19 binding domain connected to the transmembrane domain by a hinge region.

In specific embodiments of the invention, the nucleic acid construct of the invention encodes for a hu-CAR that comprises a transmembrane domain and the hinge region, wherein the transmembrane domain and the hinge region, comprise an amino acid sequence of SEQ ID NO: or a sequence with 95-99% identity thereof or is encoded by a sequence of SEQ ID NO.:106.

In specific embodiments of the invention, the nucleic acid construct of the invention encodes for a hu-CAR that comprises a costimulatory domain/a functional signaling domain derived from a protein selected from group consisting of one or more of 4-1BB, CD27, NFAT, Sirtuin1 (SIRT1) and ICAM1.

In specific embodiments of the invention, the nucleic acid construct of the invention encodes for a hu-CAR that comprises a costimulatory domain, wherein the costimulatory domain is selected from amino acid sequences of SEQ ID NO: 6, SEQ ID NO: 9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, or SEQ ID NO:34, or a sequence with 95-99% identity to an amino acid sequence of SEQ ID NO: 6, SEQ ID NO: 9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, or SEQ ID NO:34 thereof.

In specific embodiments of the invention, the nucleic acid construct of the invention encodes for a hu-CAR that comprises a costimulatory domain, wherein the costimulatory domain comprises a sequence selected from SEQ ID NO: 7, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, or SEQ ID NO:49 or a sequence with 95-99% identity to an amino acid sequence provided in SEQ ID NO: 7, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, or SEQ ID NO:49 thereof.

In specific embodiments of the invention, the nucleic acid construct of the invention encodes for a hu-CAR that comprises a intracellular signaling domain wherein the intracellular signaling domain is from human CD3 chain and comprises a sequence of Sequence ID NO.: 8 or is encoded by a sequence of SEQ ID NO.: 109.

In specific embodiments of the invention, the nucleic acid construct of the invention encodes for a hu-CAR that comprises a leader sequence wherein the leader sequence comprises an amino acid sequence of SEQ ID NO: 4 or is encoded by a sequence of SEQ ID NO.: 105.

In specific embodiments of the invention, the nucleic acid construct of the invention encodes for a hu-CAR and comprises a promoter of amino acid sequence selected from the group comprising of SEQ ID NO. 50, SEQ ID NO. 51, SEQ ID NO. 52, SEQ ID NO. 53, SEQ ID NO. 54, SEQ ID NO. 55, SEQ ID NO. 56, SEQ ID NO. 57, SEQ ID NO. 58, SEQ ID NO. 59, or SEQ ID NO. 60 or a sequence with 95-99% identity to an amino acid sequence provided in SEQ ID NO. 50, SEQ ID NO. 51, SEQ ID NO. 52, SEQ ID NO. 53, SEQ ID NO. 54, SEQ ID NO. 55, SEQ ID NO. 56, SEQ ID NO. 57, SEQ ID NO. 58, SEQ ID NO. 59, and SEQ ID NO. 60, thereof.

In specific embodiments of the invention, the nucleic acid construct of the invention encodes for a hu-CAR which is used for treating a subject having a disease associated with expression of a CD19.

The present invention is also directed to an huCAR, wherein huCAR comprises;
a. a signal sequence/leader sequence;
b. a scFv or fragment comprising a hu anti-CD19 binding domain as an antigen specific targeting region;
c. a hinge region;
d. a transmembrane domain;
e. a cytoplasmic domain comprising at least one co-stimulatory domain;
f. one intracellular signaling domain; and
g. optionally a promoter sequence selected from EF-1α or MND, and
wherein scFv or fragment comprising a hu anti-CD19 binding domain, comprises a heavy chain variable region, a light chain variable region; and wherein heavy chain variable region comprises a sequence of SEQ ID. NO.: 1, and wherein the light chain variable region comprises a sequence of SEQ ID. NO.: 2.

The present invention is also directed to an huCAR, wherein the huCAR comprises;
a. a signal sequence/leader sequence;
b. a scFv or fragment comprising a hu anti-CD19 binding domain as an antigen specific targeting region;
c. a hinge region;
d. a transmembrane domain;
e. a cytoplasmic domain comprising at least one co-stimulatory domain;
f. one intracellular signaling domain; and
g. optionally a promoter sequence selected from EF-1α or MND, and
wherein scFv or fragment comprising a hu anti-CD19 binding domain, comprises a heavy chain variable region, a light chain variable region; and
wherein the costimulatory domain is selected from amino acid sequences of SEQ ID NO: 6, SEQ ID NO: 9, SEQ ID NO: 10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, or SEQ ID NO:34, or a sequence with 95-99% identity to an amino acid sequence of SEQ ID NO: 6, SEQ ID NO: 9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, or SEQ ID NO:34.

The present invention is also directed to an huCAR, wherein huCAR comprises;
a. a signal sequence/leader sequence;
b. a scFv or fragment comprising a hu anti-CD19 binding domain as an antigen specific targeting region;
c. a hinge region;
d. a transmembrane domain;
e. a cytoplasmic domain comprising at least one co-stimulatory domain;
f. one intracellular signaling domain; and
g. optionally a promoter sequence selected from EF-1α or MND, and
wherein scFv or fragment comprising a hu anti-CD19 binding domain, comprises a heavy chain variable region, a light chain variable region; and
wherein the costimulatory domain comprises a sequence selected from SEQ ID NO: 7, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, or SEQ ID NO:49 or a sequence with 95-99% identity to an amino acid sequence provided in SEQ ID NO.:7, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49 thereof.

In specific embodiments of the invention, the hu-CAR comprises a heavy chain variable region (HCVR) and the light chain variable region (LCVR) of amino acid sequence of SEQ ID NO: 1 and SEQ ID NO: 2 respectively, or an amino acid sequence having at least one, two or three modifications but not more than 10/20 modifications of an amino acid sequence or a sequence with 95-99% identity to an amino acid sequence of SEQ ID NO 1 and SEQ ID NO: 2 thereof.

In specific embodiments of the invention, the hu-CAR comprises the scFv or fragment comprising a hu anti-CD19 binding domain, comprises a heavy chain variable region, a light chain variable region; and wherein, the encoded heavy chain variable region is attached to the encoded light chain variable region via a linker.

In specific embodiments of the invention, the hu-CAR comprises a linker that comprises an amino acid sequence of SEQ ID NO: 3 or is encoded by a sequence of SEQ ID. NO.: 104.

In specific embodiments of the invention, the hu-CAR comprises the transmembrane domain, wherein the transmembrane domain is a protein derived from a human CD8α.

In specific embodiments of the invention, the hu-CAR comprises anti-CD19 binding domain, wherein the anti-CD19 binding domain is connected to the transmembrane domain by a hinge region, and wherein the transmembrane domain and hinge region comprise an amino acid sequence of SEQ ID NO: 5 or a sequence with 95-99% identity thereof or is encoded by a sequence of SEQ ID NO.:106.

In specific embodiments of the invention, the hu-CAR comprises a costimulatory domain, wherein the costimulatory domain is selected from amino acid sequences of SEQ ID NO: 6, SEQ ID NO: 9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, or SEQ ID NO:34, or a sequence with 95-99% identity to an amino acid sequence of SEQ ID NO: 9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO.: 19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO.: 28, SEQ ID NO.: 29, SEQ ID NO.: 30, SEQ ID NO:31, SEQ ID NO:32, SEQ ID NO.: 33, and SEQ ID NO.: 34 thereof.

In specific embodiments of the invention, the hu-CAR comprises a costimulatory domain, wherein the costimulatory domain comprises a sequence selected from SEQ ID NO.: 7, SEQ ID NO.: 35, SEQ ID NO.: 36, SEQ ID NO.: 37, SEQ ID NO.: 38, SEQ ID NO.: 39, SEQ ID NO.: 40, SEQ ID NO.: 41, SEQ ID NO.: 42, SEQ ID NO.: 43, SEQ ID NO.: 44, SEQ ID NO.: 45, SEQ ID NO.: 46, SEQ ID NO.: 47, SEQ ID NO.: 48, or SEQ ID NO.: 49 or a sequence with 95-99% identity to an amino acid sequence provided in SEQ ID NO:35, SEQ ID NO:36, SEQ ID NO:37, SEQ ID NO.: 38, SEQ ID NO:39, SEQ ID NO.: 40, SEQ ID NO.: 41, SEQ ID NO.: 42, SEQ ID NO.: 43, SEQ ID NO.: 44, SEQ ID NO.: 45, SEQ ID NO.: 46, SEQ ID NO.: 47, SEQ ID NO.: 48, SEQ ID NO.: 49 thereof.

In specific embodiments of the invention, the hu-CAR comprises a intracellular signaling domain, wherein the intracellular signaling domain is from human CD3 chain and comprises a sequence of Sequence ID NO.: 8 or is encoded by a sequence of SEQ ID NO.: 109.

In specific embodiments of the invention, the hu-CAR comprises a leader sequence, wherein the leader sequence comprises an amino acid sequence of SEQ ID NO.: 4 or is encoded by a sequence of SEQ ID NO.: 105.

In specific embodiments of the invention, the hu-CAR comprises a promoter, wherein the promoter comprises an amino acid sequence selected from the group comprising of SEQ ID NO.: 50, SEQ ID NO.: 51, SEQ ID NO.: 52, SEQ ID NO. 53, SEQ ID NO.: 54, SEQ ID NO.: 55, SEQ ID NO.: 56, SEQ ID NO.: 57, SEQ ID NO.: 58, SEQ ID NO.: 59, or SEQ ID NO.: 60 or a sequence with 95-99% identity to an amino acid sequence provided in SEQ ID NO.: 50, SEQ ID NO.: 51, SEQ ID NO.: 52, SEQ ID NO.: 53, SEQ ID NO.: 54, SEQ ID NO.:55, SEQ ID NO. 56, SEQ ID NO.: 57, SEQ ID NO.: 58, SEQ ID NO. 59, and SEQ ID NO.: 60, thereof.

The present invention also relates to ORF3 of huCAR comprising sequences selected from SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO:71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101.

The present invention also relates to synthetic construct of huCAR, encoding amino acid sequences of SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49.

### Hu Anti-CD19 Domain

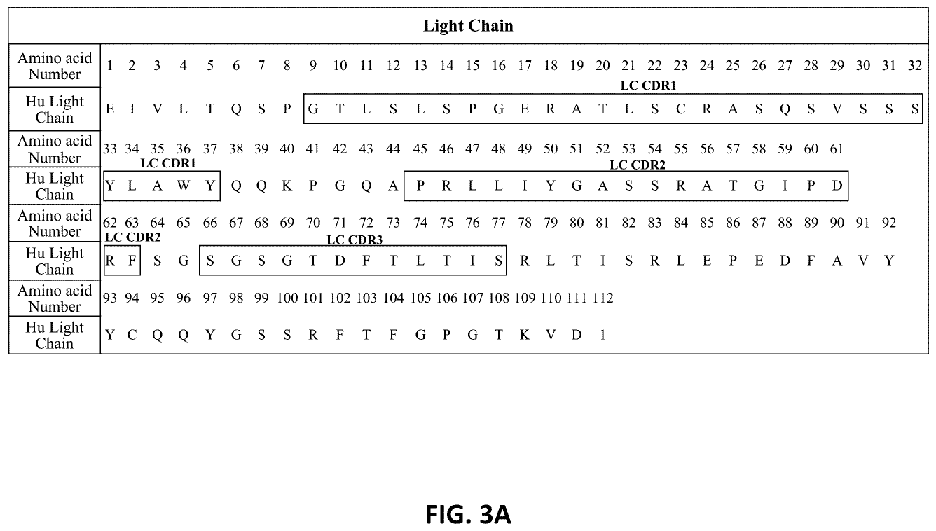

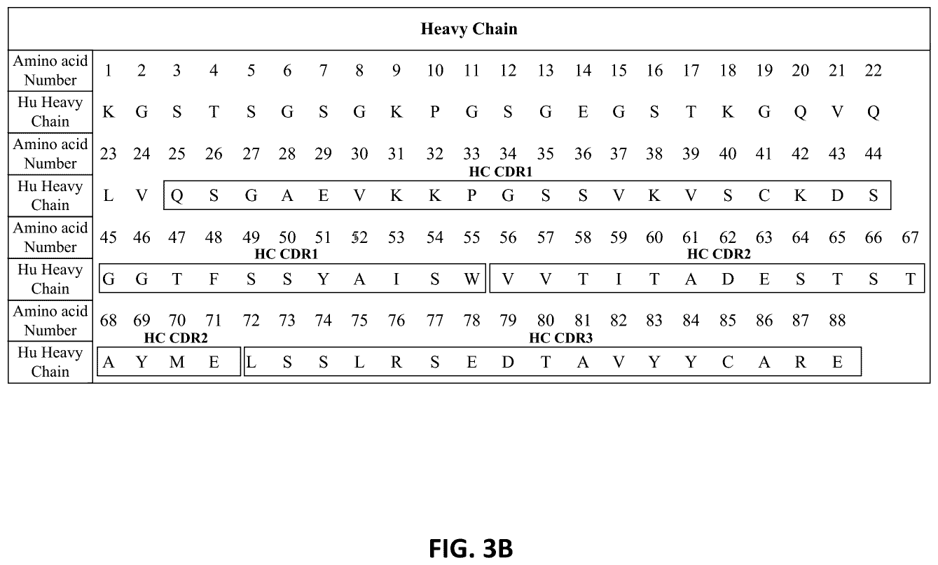

Current invention pertains to treat CD19 associated B cell malignancies where CD19 serves as an target antigen for the antigen binding domain in CAR. During maturation and development of B cells, they express various proteins on their surface and CD19 being one of them which is also expressed in most of the B cell malignancies but are absent in hematopoietic stem cells. Therefore, the cytotoxic effects are minimized by appropriate choice of antigens such as CD19. Anti CD19 CAR transduced into T cells are directed against the CD19 antigen expressing cells and effectively eliminate them without affecting the normal healthy cells. Majority of CARs developed till date have murine derived monoclonal antibody fragments as the antigen binding domain. The non-human origin CD-19 binding domain is present in most of the CAR-T cell products available commercially or in clinical trials which leads to immunogenicity of CAR-T cells. As a result, post infusion of engineered CAR expressing T cells are recognized as foreign antigen and triggers immune response to produce antibodies against CAR. Thus the non-human fragments in CAR is cleared prematurely hindering the efficacy of CAR-T cells and limited the repeat dosages. Therefore, hu anti-CD19 binding domain is designed from the fragment of KIG1 to obtain the single chain variable fragment (scFv) against the CD19 as known in prior art. Binding domains comprises of the complementarity determining regions (CDRs) and heavy and light chain frameworks as given in the FIGS. 3A-3B. Usually, there are three CDRs namely CDR1, CDR2 and CDR3 which combined together forms a hypervariable region identifying the target antigen. Furthermore, the hu anti-CD19 CAR will overcome the obstacle of immunogenicity and minimize the adverse side effects of CAR-T cell product improving its efficacy and safety.

In the present invention, the hu anti-CD19 binding domain has heavy chain framework region of SEQ ID NO: 102 and Light chain framework region of SEQ ID NO: 103. In another aspect, the hu anti-CD19 binding domain has heavy chain CDR1 (HC CDR1), heavy chain CDR2 (LC CDR2) and heavy chain CDR3 (HC CDR3) as shown in FIG. 3B.

In further aspect, hu anti-CD19 binding domain (scFv) has a variable heavy chain region VH) and a variable light chain region (VL). VH of the scFv in the present invention is a polypeptide sequence represented by SEQ ID NO 1 having complementarity specific to T cells. Similarly, VL region is the amino acid sequence represented by SEQ ID NO: 2 figured to have high identity to framework regions of the human light chain and CDRs sequence.

In another aspect, the hu anti-CD19 binding domain has light chain CDR1 (LC CDR1), light chain CDR2 (LC CDR2) and light chain CDR3 (LC CDR3) as shown in FIG. 3A.

The CAR's ScFv (single-chain variable fragment) region binds to the target antigen through a combination of cation-pi interactions. The ScFv region contains aromatic residues e.g., phenylalanine (Phe, F), tyrosine (Tyr, Y), or tryptophan (Trp, W). that participate in cation-pi interactions with positively charged residues e.g., lysine (Lys, K) or arginine (Arg, R) on the target huCD19 antigen. The cation-pi interaction helps stabilize the binding of the CAR to the target antigen, increasing the binding affinity and specificity, and therapeutic efficacy.

For instance, the single chain variable fragment (ScFV) SEQ ID NO:2 huantiCD19 light chain binds with its—TYR-33 to huCD19 antigen ARG-250 at 3.8 Å. Also, single chain variable fragment (ScFV) SEQ ID NO:2 huanti-CD19 Light chain variable region binds with its TRP-50 to huCD19 antigen ARG-277 at 5.7 Å. The single chain variable fragment (ScFV) SEQ ID NO:2 huantiCD19 light chain binds with its TRP-50 to huCD19 antigen ARG-286 at 4.7 Å. Also the single chain variable fragment (ScFV) SEQ ID NO:2 huantiCD19 light chain binds with its TYR-92 to huCD19 antigen ARG-250 at 4.5 Å. The single chain variable fragment (ScFV) SEQ ID NO:2 huantiCD19 light chain binds with its TYR-96 to huCD19 antigen TRP-81 at 4.0 Å. The single chain variable fragment (ScFV) SEQ ID NO:2 huantiCD19 light chain binds with its PHE-97 to huCD19 antigen ARG-76 at 4.2 Å.

In the present invention, the leader sequence at the amino terminal is an amino acid sequence of SEQ ID NO: 4 and is encoded by the mRNA sequence selected from SEQ ID NO: 105 or mRNA sequence with at least 95% identity from the parent SEQ ID NO:105.

### Hinge and Transmembrane Domain

The antigen binding domain of CAR is connected to the transmembrane domain via a hinge region. The hinge region is derived from CD8α similar to the prior art. Anchorage between extracellular domain and intracellular domain is facilitated by the transmembrane domain in the synthetic CAR construct developed in the present invention. Generally, the transmembrane domain is chosen from type I proteins such as CDζ, CD28, CD8a and also other T cell related molecules.

In the present invention, transmembrane domain derived from human CD8α comprises of amino acid sequence of SEQ ID NO: 5.

### Co-Stimulatory and Intracellular Signaling Domain

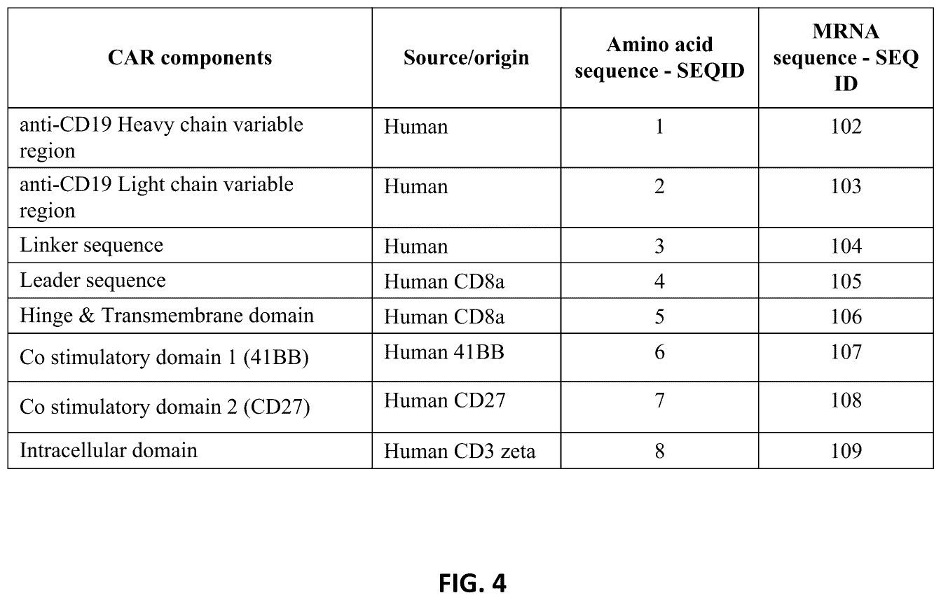

Intracellular domain of the synthetic CAR construct in present invention has two domains, viz., co-stimulatory domain and intracellular signaling domain derived from cytoplasmic domains of T cells receptor complex. Effector function and anti-tumor activity is influenced by the choice of intracellular domain and expressed by the cells encoding the CAR majorly T cells expressing CAR. In one embodiment, the cytoplasmic domain is derived from human CDζ (comprising of amino acid sequences as in FIG. 4.

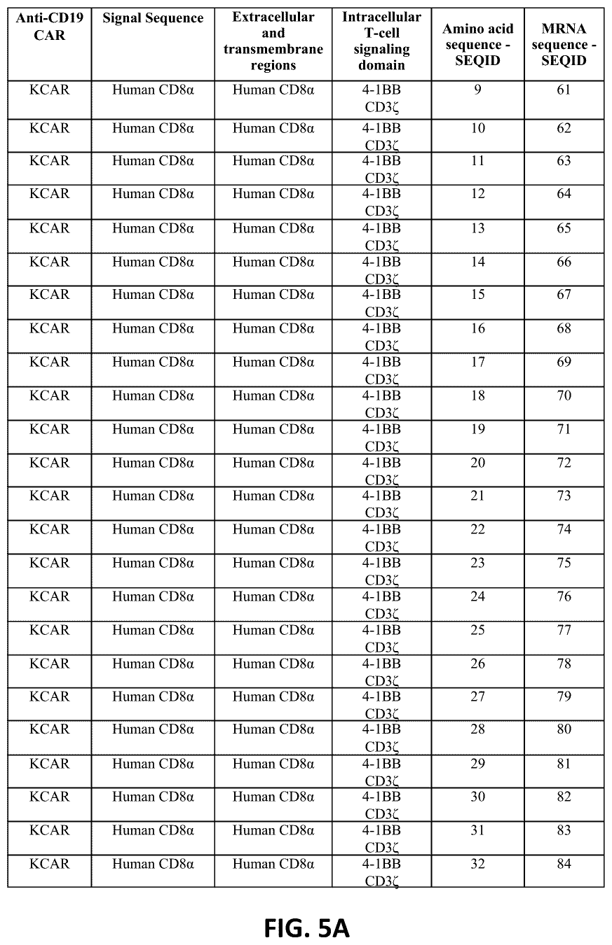

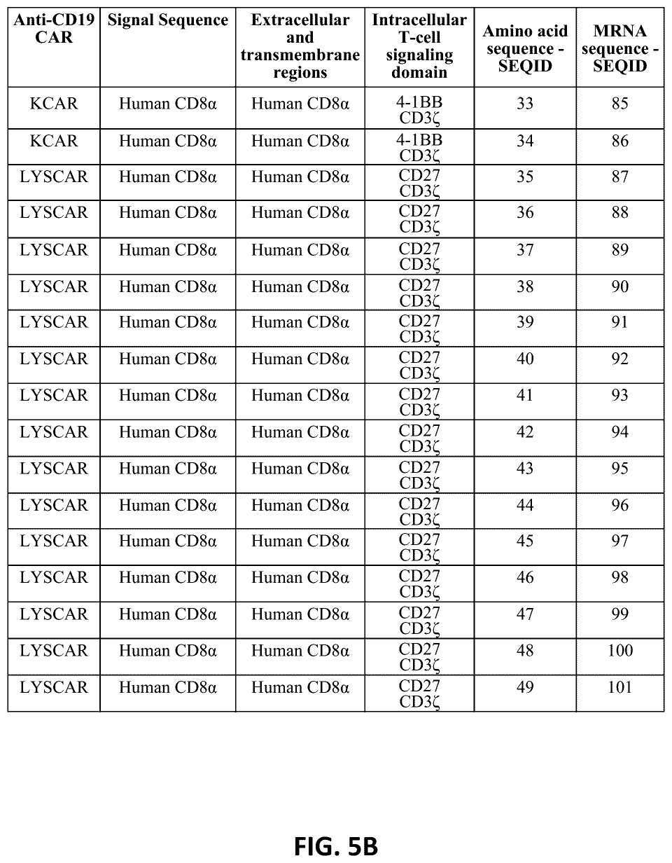

In another embodiment, the amino acid and nucleic sequence for recombinant synthetic CAR construct comprising human 4-1BB and or CD27 as co-stimulatory domain is selected from FIG. 5.

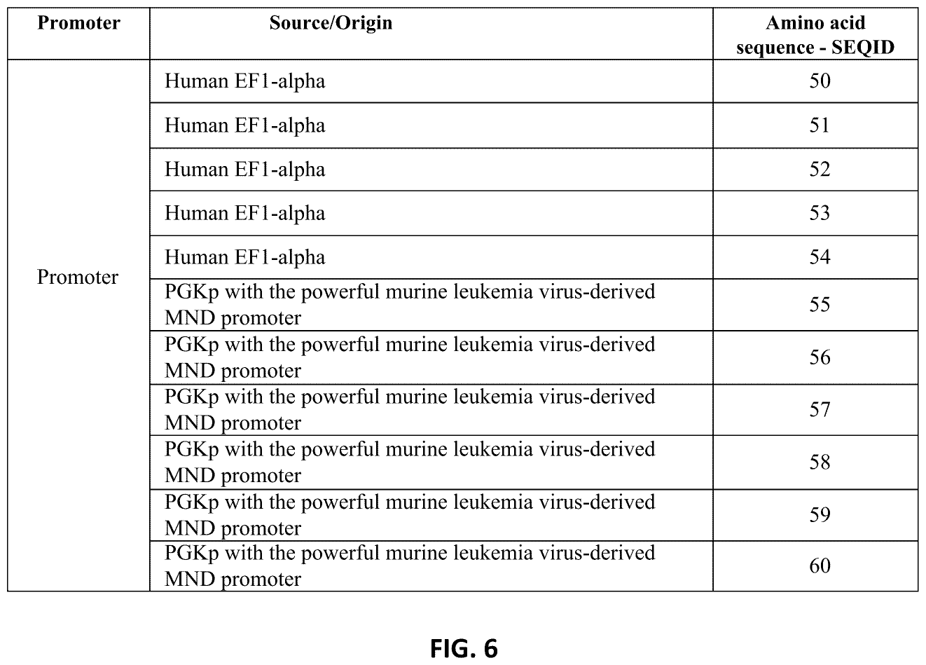

In another embodiment, the human EF-1α and MND promoter sequences are selected from the FIG. 6.

The inclusion of the 4-1BB co-stimulatory domain in the CAR structure enhances the persistence, proliferation, and anti-tumor activity of CAR-T cells. Co-stimulation through 4-1BB provides an additional signal that complements the primary activation signal delivered by the TCR, leading to more robust and sustained T-cell activation. The activation of 4-1BB also plays a role in the regulation of certain cytokines and immune mediators, further influencing the overall T-cell response. CD27 has been reported to activate the non-canonical NF-κB signaling pathway through its interaction with TRAF2. CD27 signaling plays a role in enhancing T-cell and B-cell activation, proliferation, and differentiation by production of cytokines and effector molecules, which are critical for mounting an effective immune response against pathogens or antigens. It can also influence apoptosis through several mechanisms, including the regulation of caspase activity and the expression of pro-survival or pro-apoptotic molecules. This pathway is critical for supporting the metabolic demands of activated immune cells and maintaining their long-term survival, particularly in memory T cell development.

In another embodiment, the co-stimulatory domain of cytoplasmic domain is derived from human 4-1BB is labelled as KCAR in the present invention and encoded by mRNA sequence selected from group of sequences of SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85 and SEQ ID NO: 86 or a mRNA sequence with at least 95% identity to an mRNA sequence provided in SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85 and SEQ ID NO: 86 thereof.

In another embodiment, the co-stimulatory domain of cytoplasmic domain is derived from human CD27 is labelled as LYSCAR in the present invention and encoded by mRNA sequence selected from group of sequences of SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO:100 and SEQ ID NO: 101 or a mRNA sequence with at least 95% identity to an mRNA sequence provided in SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO:100 and SEQ ID NO: 101 thereof.

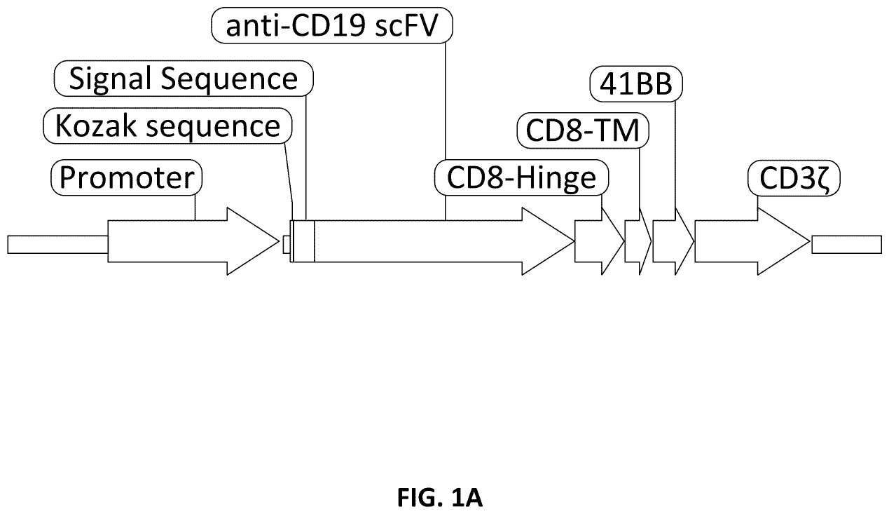

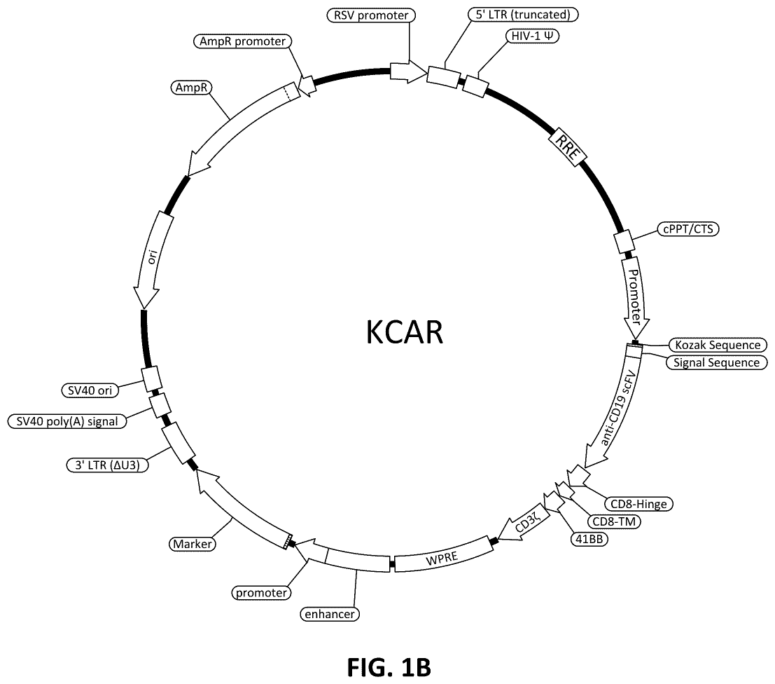

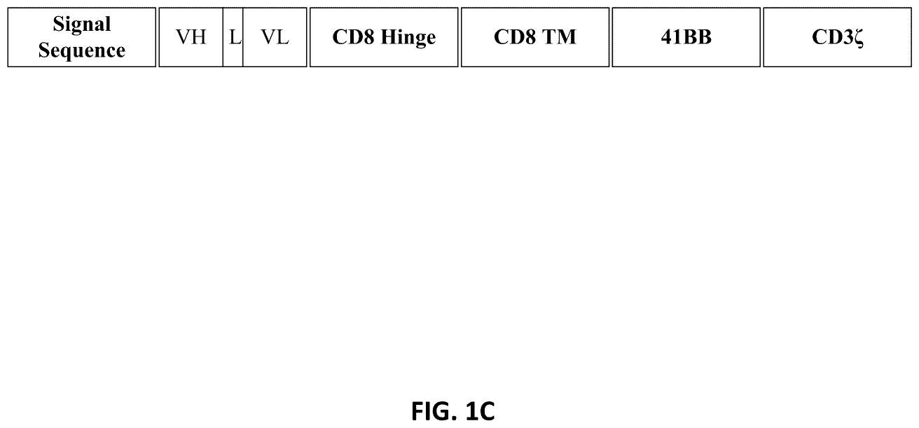

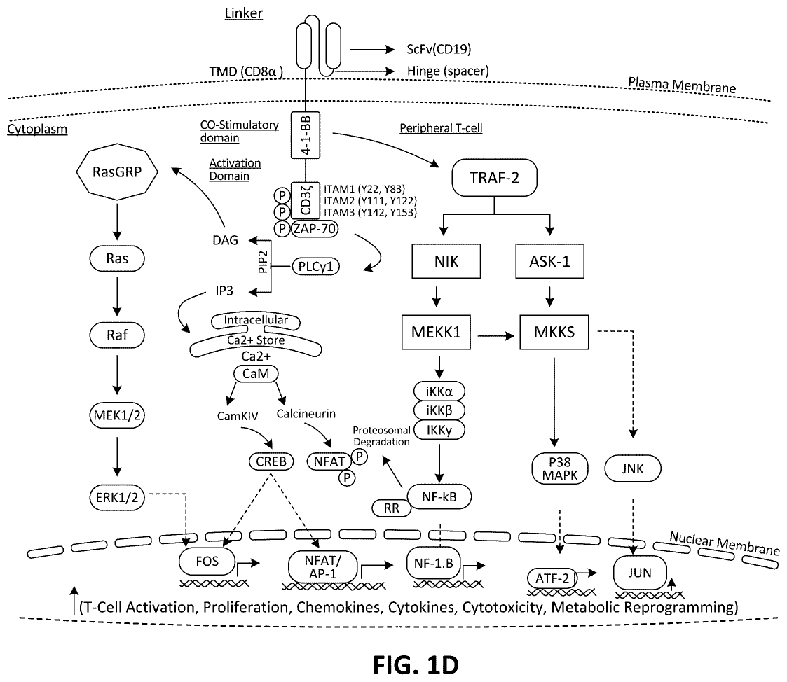

FIGS. 1A and 1B provide an exemplary Synthetic construct of hu anti-CD19-4-1BBZ (KCAR). FIG. 1C also provides a schematic representation of the construct and FIG. 1D is its predictive signaling network

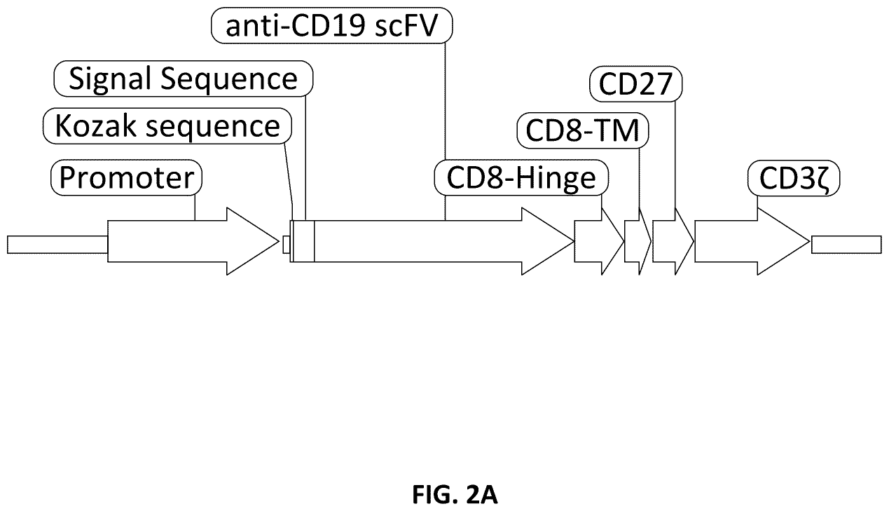

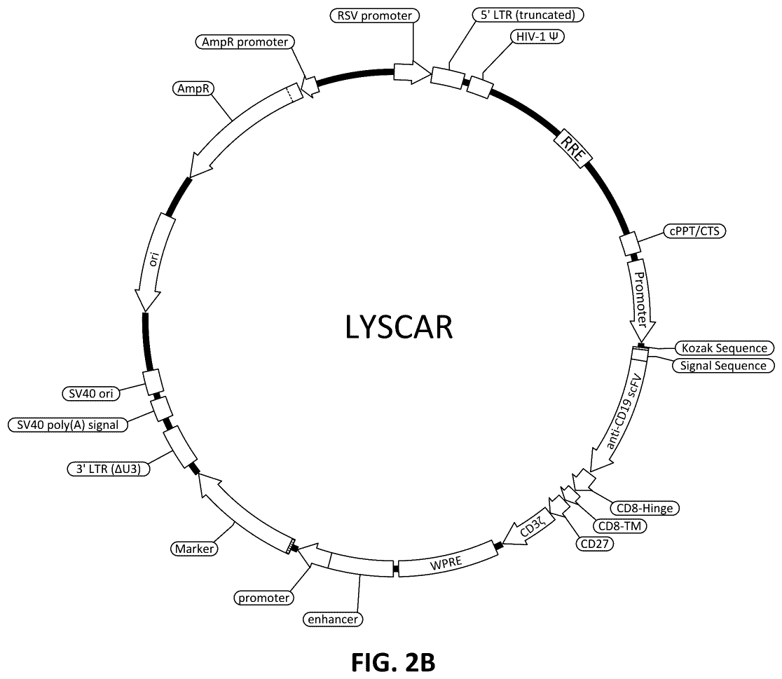

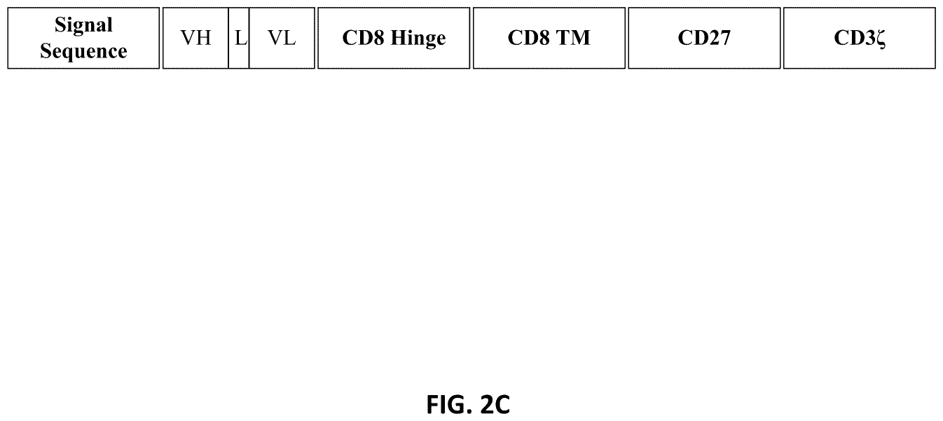

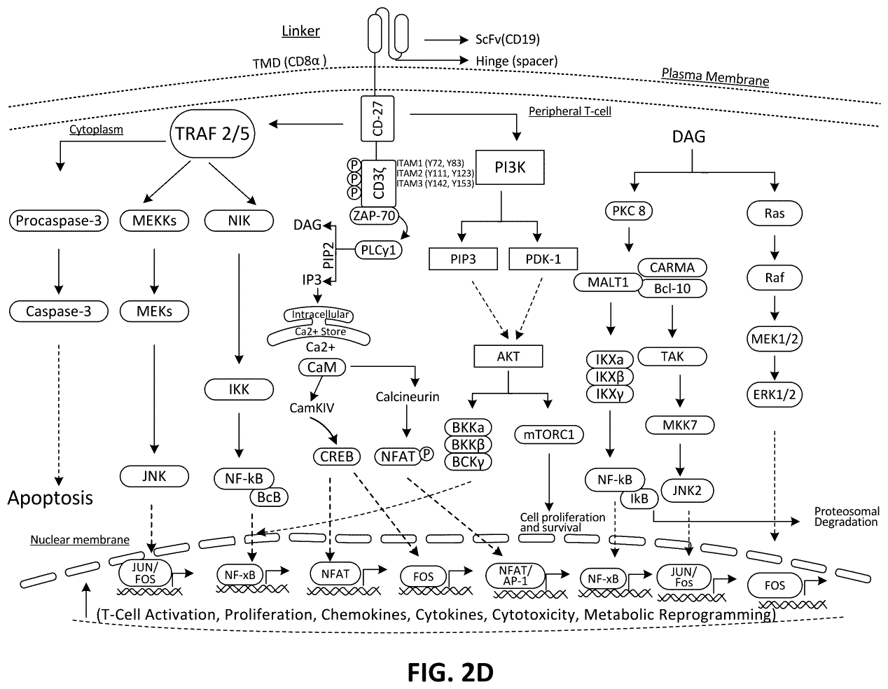

Similarly, FIGS. 2A and 2B Provide a synthetic construct of hu anti-CD19-CD27Z (LYSCAR). FIG. 2C provides a schematic representation of the construct and FIG. 2D is its predictive signaling network.

What is claimed is:

1-25. (canceled)

26. A nucleic acid encoding huCAR, wherein the huCAR comprises:
a) a signal sequence/leader sequence;
b) a scFv or fragment comprising a hu anti-CD19 binding domain as an antigen specific targeting region;
c) a hinge region;
d) a transmembrane domain;
e) a cytoplasmic domain comprising at least one co-stimulatory domain;
f) one intracellular signaling domain; and
g) optionally a promoter sequence selected from EF-1α or MND,
wherein the scFv or fragment comprising the hu anti-CD19 binding domain comprises a heavy chain variable region and a light chain variable region,
wherein the heavy chain variable region comprises a sequence of SEQ ID NO:1, and
wherein the light chain variable region comprises a sequence of SEQ ID NO:2.

27. The nucleic acid construct as claimed in claim 26, which comprises a heavy chain variable region encoding framework, a light chain variable region encoding framework; and wherein the heavy chain encoding framework region comprises a sequence of SEQ ID NO:102, and wherein the light chain encoding framework region comprises a sequence of SEQ ID NO:103.

28. The nucleic acid construct as claimed in claim 26, wherein the heavy chain variable region (HCVR) and the light chain variable region (LCVR) comprises an amino acid sequence having at least one, two or three modifications but not more than 10/20 modifications of an amino acid sequence or a sequence with 95-99% identity to an amino acid sequence of SEQ ID NO:1 and SEQ ID NO:2 thereof.

29. The nucleic acid construct as claimed in claim 26, wherein a linker comprises an amino acid sequence of SEQ ID NO:3 or is encoded by a sequence of SEQ ID NO:104.

30. The nucleic acid construct as claimed in claim 26, wherein the anti-CD19 binding domain is connected to the transmembrane domain by a hinge region.

31. The nucleic acid construct as claimed in claim 30, wherein the transmembrane domain and the hinge region comprise an amino acid sequence of SEQ ID NO:5 or a sequence with 95-99% identity thereof or is encoded by a sequence of SEQ ID NO:106.

32. The nucleic acid construct or a synthetic construct as claimed in claim 26, wherein the co-stimulatory domain is a functional signaling domain derived from a human of a protein selected from group consisting of one or more of 4-1BB, CD27, NFAT, Sirtuin1 (SIRT1) and ICAM1.

33. The nucleic acid construct as claimed in claim 32, wherein the co-stimulatory domain is selected from any one or more amino acid sequences of SEQ ID NO:6, SEQ ID NO: 9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, or SEQ ID NO:34, or a sequence with 95-99% identity to an amino acid sequence of SEQ ID NO:6, SEQ ID NO: 9, SEQ ID NO:10, SEQ ID NO: 11, SEQ ID NO:12, SEQ ID NO:13, SEQ ID NO:14, SEQ ID NO:15, SEQ ID NO: 16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, or SEQ ID NO:34.

34. The nucleic acid construct as claimed in claim 32, wherein the co-stimulatory domain comprises a sequence selected from any one or more of SEQ ID NO: 7, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, or SEQ ID NO:49, a sequence with 95-99% identity to an amino acid sequence provided in SEQ ID NO: 7, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO 44, SEQ ID NO:45, SEQ ID NO:46, SEQ ID NO:47, SEQ ID NO:48, or SEQ ID NO49.

35. The nucleic acid construct as claimed in claim 26, wherein the intracellular signaling domain is from human CDζ (chain and comprises a sequence of SEQ ID NO:8 or is encoded by a sequence of SEQ ID NO:109 or a sequence with 95-99% identity thereof.

36. The nucleic acid construct as claimed in claim 26, wherein the leader sequence comprises an amino acid sequence of SEQ ID NO:4 or is encoded by a sequence of SEQ ID NO:105.

37. The nucleic acid construct as claimed in claim 26, wherein the promoter is of an amino acid sequence selected from the group consisting of any one or more of SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, or SEQ ID NO:60, a sequence with 95-99% identity to an amino acid sequence provided in SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, or SEQ ID NO:60.

38. The nucleic acid construct as claimed in claim 26, wherein the huCAR comprises sequences selected from any one or more of SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO:71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, or SEQ ID NO: 101.

39. A nucleic acid construct as claimed in claim 26, used for treating a subject having a disease associated with expression of a CD19.

40. A huCAR, wherein the huCAR comprises;
a. a signal sequence/leader sequence;
b. a scFv or fragment comprising a hu anti-CD19 binding domain as an antigen specific targeting region;
c. a hinge region;
d. a transmembrane domain;
e. a cytoplasmic domain comprising at least one co-stimulatory domain;
f. one intracellular signaling domain; and
g. optionally a promoter sequence selected from EF-1α or MND,
wherein the scFv or fragment comprising the hu anti-CD19 binding domain comprises a heavy chain variable region and a light chain variable region,
wherein heavy chain variable region comprises a sequence of SEQ ID. NO.: 1, and
wherein the light chain variable region comprises a sequence of SEQ ID. NO.: 2.

41. The huCAR as claimed in claim 40, wherein the heavy chain variable region (HCVR) and the light chain variable region (LCVR) comprise an amino acid sequence of SEQ ID NO: 1 and SEQ ID NO: 2 or an amino acid sequence having at least one, two or three modifications but not more than 10/20 modifications of an amino acid sequence or a sequence with 95-99% identity to an amino acid sequence of SEQ ID NO 1 and SEQ ID NO: 2 thereof.

42. The huCAR as claimed in claim 40, wherein a linker comprises an amino acid sequence of SEQ ID NO: 3 or is encoded by a sequence of SEQ ID. NO.: 104.

43. The huCAR as claimed in claim 40, wherein the anti-CD19 binding domain is connected to the transmembrane domain by a hinge region, and wherein the transmembrane domain and hinge region comprise an amino acid sequence of SEQ ID NO: 5 or a sequence with 95-99% identity thereof or is encoded by a sequence of SEQ ID NO.:106.

44. The huCAR as claimed in claim 40, wherein a co-stimulatory domain is selected from amino acid sequences consisting of any one or more of SEQ ID NO: 6, SEQ ID NO: 9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, or SEQ ID NO:34, or a sequence with 95-99% identity to an amino acid sequence of SEQ ID NO: 9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO.: 19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO.: 28, SEQ ID NO.: 29, SEQ ID NO.: 30, SEQ ID NO:31, SEQ ID NO:32, SEQ ID NO.: 33, or SEQ ID NO.: 34.

45. The huCAR as claimed in claim 40, wherein a co-stimulatory domain comprises a sequence selected from any one or more of SEQ ID NO.: 7, SEQ ID NO.: 35, SEQ ID NO.: 36, SEQ ID NO.: 37, SEQ ID NO.: 38, SEQ ID NO.: 39, SEQ ID NO.: 40, SEQ ID NO.: 41, SEQ ID NO.: 42, SEQ ID NO.: 43, SEQ ID NO.: 44, SEQ ID NO.: 45, SEQ ID NO.: 46, SEQ ID NO.: 47, SEQ ID NO.: 48, or SEQ ID NO.: 49 or a sequence with 95-99% identity to an amino acid sequence provided in SEQ ID NO:35, SEQ ID NO:36, SEQ ID NO:37, SEQ ID NO.: 38, SEQ ID NO:39, SEQ ID NO.: 40, SEQ ID NO.: 41, SEQ ID NO.: 42, SEQ ID NO.: 43, SEQ ID NO.: 44, SEQ ID NO.: 45, SEQ ID NO.: 46, SEQ ID NO.: 47, SEQ ID NO.: 48, or SEQ ID NO.: 49.

46. A huCAR as claimed in claim 40, wherein the intracellular signaling domain is from human CDζ (chain and comprises a sequence of Sequence ID NO.: 8 or is encoded by a sequence of SEQ ID NO.: 109.

47. A huCAR as claimed in claim 40, wherein the leader sequence comprises an amino acid sequence of SEQ ID NO.: 4 or is encoded by a sequence of SEQ ID NO.: 105.

48. A huCAR as claimed in claim 40, wherein the promoter comprises an amino acid sequence selected from any one or more of SEQ ID NO.: 50, SEQ ID NO.: 51, SEQ ID NO.: 52, SEQ ID NO. 53, SEQ ID NO.: 54, SEQ ID NO.: 55, SEQ ID NO.: 56, SEQ ID NO.: 57, SEQ ID NO.: 58, SEQ ID NO.: 59, or SEQ ID NO.: 60 or a sequence with 95-99% identity to an amino acid sequence provided in SEQ ID NO.: 50, SEQ ID NO.: 51, SEQ ID NO.: 52, SEQ ID NO.: 53, SEQ ID NO.: 54, SEQ ID NO.:55, SEQ ID NO. 56, SEQ ID NO.: 57, SEQ ID NO.: 58, SEQ ID NO. 59, or SEQ ID NO.: 60.

49. A huCAR, wherein the huCAR comprises;
a. a signal sequence/leader sequence;
b. a scFv or fragment comprising a hu anti-CD19 binding domain as an antigen specific targeting region;
c. a hinge region;
d. a transmembrane domain;
e. a cytoplasmic domain comprising at least one co-stimulatory domain;
f. one intracellular signaling domain; and
g. optionally a promoter sequence selected from EF-1α or MND,
wherein the scFv or fragment comprising the hu anti-CD19 binding domain comprises a heavy chain variable region and a light chain variable region,
wherein the heavy chain variable region comprises a sequence of SEQ ID. NO.: 1, and the light chain variable region comprises a sequence of SEQ ID. NO.: 2,
wherein a linker comprises an amino acid sequence of SEQ ID NO: 3,
wherein the anti-CD19 binding domain is connected to the transmembrane domain by a hinge region,
wherein the transmembrane domain and hinge region comprise an amino acid sequence of SEQ ID NO: 5 or a sequence with 95-99% identity thereof,
wherein the co-stimulatory domain includes amino acid sequences selected from any one or more of SEQ ID NO: 6, SEQ ID NO: 9, SEQ ID NO:10, SEQ ID NO:11, SEQ ID NO: 12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO.: 7, SEQ ID NO.: 35, SEQ ID NO.: 36, SEQ ID NO.: 37, SEQ ID NO.: 38, SEQ ID NO.: 39, SEQ ID NO.: 40, SEQ ID NO.: 41, SEQ ID NO.: 42, SEQ ID NO.: 43, SEQ ID NO.: 44, SEQ ID NO.: 45, SEQ ID NO.: 46, SEQ ID NO.: 47, SEQ ID NO.: 48, or SEQ ID NO.: 49 or a sequence with 95-99% identity to an amino acid sequence of SEQ ID NO: 9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO.: 19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO.: 28, SEQ ID NO.: 29, SEQ ID NO.: 30, SEQ ID NO:31, SEQ ID NO:32, SEQ ID NO.: 33, SEQ ID NO.: 34, SEQ ID NO.: 7, SEQ ID NO.: 35, SEQ ID NO.: 36, SEQ ID NO.: 37, SEQ ID NO.: 38, SEQ ID NO.: 39, SEQ ID NO.: 40, SEQ ID NO.: 41, SEQ ID NO.: 42, SEQ ID NO.: 43, SEQ ID NO.: 44, SEQ ID NO.: 45, SEQ ID NO.: 46, SEQ ID NO.: 47, SEQ ID NO.: 48, or SEQ ID NO.: 49,
wherein the intracellular signaling domain comprises a sequence of Sequence ID NO.: 8;
wherein the leader sequence comprises an amino acid sequence of SEQ ID NO.: 4, and
wherein the promoter comprises an amino acid sequence selected from any one or more of SEQ ID NO.: 50, SEQ ID NO.: 51, SEQ ID NO.: 52, SEQ ID NO. 53, SEQ ID NO.: 54, SEQ ID NO.: 55, SEQ ID NO.: 56, SEQ ID NO.: 57, SEQ ID NO.: 58, SEQ ID NO.: 59, or SEQ ID NO.: 60 or a sequence with 95-99% identity to an amino acid sequence provided in SEQ ID NO.: 50, SEQ ID NO.: 51, SEQ ID NO.: 52, SEQ ID NO.: 53, SEQ ID NO.: 54, SEQ ID NO.:55, SEQ ID NO. 56, SEQ ID NO.: 57, SEQ ID NO.: 58, SEQ ID NO. 59, or SEQ ID NO.: 60.


SEQUENCE LISTING (109 sequences)

SEQ ID NO: 1 - protein, 69 aa, synthetic construct
QVQLVQSGAEVKKPGSSVKVSCKDSGGTFSSYAISWVVTITADESTSTAYMELSSLRSEDTAVYYCARE

SEQ ID NO: 2 - protein, 105 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKV

SEQ ID NO: 3 - protein, 21 aa, synthetic construct
DIKGSTSGSGKPGSGEGSTKG

SEQ ID NO: 4 - protein, 30 aa, synthetic construct
MALPVTALLLPLALLLHAARPSQFRVSPLD

SEQ ID NO: 5 - protein, 83 aa, synthetic construct
FVPVFLPAKPTTTPAPRPPTPAPTIASQPLSLRPEACRPAAGGAVHTRGLDFACDIYIWAPLAGTCGVLLLSLVITLYCNHRN

SEQ ID NO: 6 - protein, 35 aa, synthetic construct
VVKRGRKKLLYIFKQPFMRPVQTTQEEDGCSCRFP

SEQ ID NO: 7 - protein, 48 aa, synthetic construct
QRRKYRSNKGESPVEPAEPCRYSCPREEEGSTIPIQEDYRKPEPACSP

SEQ ID NO: 8 - protein, 39 aa, synthetic construct
RVKFSRSADAPAYQQGQNQLYNELNLGRREEYDVLDKRR

SEQ ID NO: 9 - protein, 376 aa, synthetic construct
MALPVTALLLPLALLLHAARPSQFRVSPLDEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVVTITADESTSTAYMELSSLRSEDTAVYYCAREFVPVFLPAKPTTTPAPRPPTPAPTIASQPLSLRPEACRPAAGGAVHTRGLDFACDIYIWAPLAGTCGVLLLSLVITLYCNHRNQRRRFSVVKRGRKKLLYIFKQPFMRPVQTTQEEDGCSCRFPRVKFSRSADAPAYQQGQNQLYNELNLG

SEQ ID NO: 10 - protein, 377 aa, synthetic construct
MALPVTALLLPLALLLHAARPSQFRVSPLDEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVVTITADESTSTAYMELSSLRSEDTAVYYCAREFVPVFLPAKPTTTPAPRPPTPAPTIASQPLSLRPEACRPAAGGAVHTRGLDFACDIYIWAPLAGTCGVLLLSLVITLYCNHRNQRRRFSVVKRGRKKLLYIFKQPFMRPVQTTQEEDGCSCRFPRVKFSRSADAPAYQQGQNQLYNELNLGR

SEQ ID NO: 11 - protein, 378 aa, synthetic construct
MALPVTALLLPLALLLHAARPSQFRVSPLDEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVVTITADESTSTAYMELSSLRSEDTAVYYCAREFVPVFLPAKPTTTPAPRPPTPAPTIASQPLSLRPEACRPAAGGAVHTRGLDFACDIYIWAPLAGTCGVLLLSLVITLYCNHRNQRRRFSVVKRGRKKLLYIFKQPFMRPVQTTQEEDGCSCRFPRVKFSRSADAPAYQQGQNQLYNELNLGRR

SEQ ID NO: 12 - protein, 379 aa, synthetic construct
MALPVTALLLPLALLLHAARPSQFRVSPLDEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVVTITADESTSTAYMELSSLRSEDTAVYYCAREFVPVFLPAKPTTTPAPRPPTPAPTIASQPLSLRPEACRPAAGGAVHTRGLDFACDIYIWAPLAGTCGVLLLSLVITLYCNHRNQRRRFSVVKRGRKKLLYIFKQPFMRPVQTTQEEDGCSCRFPRVKFSRSADAPAYQQGQNQLYNELNLGRRE

SEQ ID NO: 13 - protein, 380 aa, synthetic construct
MALPVTALLLPLALLLHAARPSQFRVSPLDEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVVTITADESTSTAYMELSSLRSEDTAVYYCAREFVPVFLPAKPTTTPAPRPPTPAPTIASQPLSLRPEACRPAAGGAVHTRGLDFACDIYIWAPLAGTCGVLLLSLVITLYCNHRNQRRRFSVVKRGRKKLLYIFKQPFMRPVQTTQEEDGCSCRFPRVKFSRSADAPAYQQGQNQLYNELNLGRREE

SEQ ID NO: 14 - protein, 381 aa, synthetic construct
MALPVTALLLPLALLLHAARPSQFRVSPLDEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVVTITADESTSTAYMELSSLRSEDTAVYYCAREFVPVFLPAKPTTTPAPRPPTPAPTIASQPLSLRPEACRPAAGGAVHTRGLDFACDIYIWAPLAGTCGVLLLSLVITLYCNHRNQRRRFSVVKRGRKKLLYIFKQPFMRPVQTTQEEDGCSCRFPRVKFSRSADAPAYQQGQNQLYNELNLGRREEY

SEQ ID NO: 15 - protein, 382 aa, synthetic construct
MALPVTALLLPLALLLHAARPSQFRVSPLDEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVVTITADESTSTAYMELSSLRSEDTAVYYCAREFVPVFLPAKPTTTPAPRPPTPAPTIASQPLSLRPEACRPAAGGAVHTRGLDFACDIYIWAPLAGTCGVLLLSLVITLYCNHRNQRRRFSVVKRGRKKLLYIFKQPFMRPVQTTQEEDGCSCRFPRVKFSRSADAPAYQQGQNQLYNELNLGRREEYD

SEQ ID NO: 16 - protein, 383 aa, synthetic construct
MALPVTALLLPLALLLHAARPSQFRVSPLDEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVVTITADESTSTAYMELSSLRSEDTAVYYCAREFVPVFLPAKPTTTPAPRPPTPAPTIASQPLSLRPEACRPAAGGAVHTRGLDFACDIYIWAPLAGTCGVLLLSLVITLYCNHRNQRRRFSVVKRGRKKLLYIFKQPFMRPVQTTQEEDGCSCRFPRVKFSRSADAPAYQQGQNQLYNELNLGRREEYDV

SEQ ID NO: 17 - protein, 384 aa, synthetic construct
MALPVTALLLPLALLLHAARPSQFRVSPLDEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVVTITADESTSTAYMELSSLRSEDTAVYYCAREFVPVFLPAKPTTTPAPRPPTPAPTIASQPLSLRPEACRPAAGGAVHTRGLDFACDIYIWAPLAGTCGVLLLSLVITLYCNHRNQRRRFSVVKRGRKKLLYIFKQPFMRPVQTTQEEDGCSCRFPRVKFSRSADAPAYQQGQNQLYNELNLGRREEYDVL

SEQ ID NO: 18 - protein, 385 aa, synthetic construct
MALPVTALLLPLALLLHAARPSQFRVSPLDEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVVTITADESTSTAYMELSSLRSEDTAVYYCAREFVPVFLPAKPTTTPAPRPPTPAPTIASQPLSLRPEACRPAAGGAVHTRGLDFACDIYIWAPLAGTCGVLLLSLVITLYCNHRNQRRRFSVVKRGRKKLLYIFKQPFMRPVQTTQEEDGCSCRFPRVKFSRSADAPAYQQGQNQLYNELNLGRREEYDVLD

SEQ ID NO: 19 - protein, 386 aa, synthetic construct
MALPVTALLLPLALLLHAARPSQFRVSPLDEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVVTITADESTSTAYMELSSLRSEDTAVYYCAREFVPVFLPAKPTTTPAPRPPTPAPTIASQPLSLRPEACRPAAGGAVHTRGLDFACDIYIWAPLAGTCGVLLLSLVITLYCNHRNQRRRFSVVKRGRKKLLYIFKQPFMRPVQTTQEEDGCSCRFPRVKFSRSADAPAYQQGQNQLYNELNLGRREEYDVLDK

SEQ ID NO: 20 - protein, 387 aa, synthetic construct
MALPVTALLLPLALLLHAARPSQFRVSPLDEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVVTITADESTSTAYMELSSLRSEDTAVYYCAREFVPVFLPAKPTTTPAPRPPTPAPTIASQPLSLRPEACRPAAGGAVHTRGLDFACDIYIWAPLAGTCGVLLLSLVITLYCNHRNQRRRFSVVKRGRKKLLYIFKQPFMRPVQTTQEEDGCSCRFPRVKFSRSADAPAYQQGQNQLYNELNLGRREEYDVLDKR

SEQ ID NO: 21 - protein, 388 aa, synthetic construct
MALPVTALLLPLALLLHAARPSQFRVSPLDEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVVTITADESTSTAYMELSSLRSEDTAVYYCAREFVPVFLPAKPTTTPAPRPPTPAPTIASQPLSLRPEACRPAAGGAVHTRGLDFACDIYIWAPLAGTCGVLLLSLVITLYCNHRNQRRRFSVVKRGRKKLLYIFKQPFMRPVQTTQEEDGCSCRFPRVKFSRSADAPAYQQGQNQLYNELNLGRREEYDVLDKRR

SEQ ID NO: 22 - protein, 389 aa, synthetic construct
MALPVTALLLPLALLLHAARPSQFRVSPLDEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVVTITADESTSTAYMELSSLRSEDTAVYYCAREFVPVFLPAKPTTTPAPRPPTPAPTIASQPLSLRPEACRPAAGGAVHTRGLDFACDIYIWAPLAGTCGVLLLSLVITLYCNHRNQRRRFSVVKRGRKKLLYIFKQPFMRPVQTTQEEDGCSCRFPRVKFSRSADAPAYQQGQNQLYNELNLGRREEYDVLDKRRG

SEQ ID NO: 23 - protein, 390 aa, synthetic construct
MALPVTALLLPLALLLHAARPSQFRVSPLDEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVVTITADESTSTAYMELSSLRSEDTAVYYCAREFVPVFLPAKPTTTPAPRPPTPAPTIASQPLSLRPEACRPAAGGAVHTRGLDFACDIYIWAPLAGTCGVLLLSLVITLYCNHRNQRRRFSVVKRGRKKLLYIFKQPFMRPVQTTQEEDGCSCRFPRVKFSRSADAPAYQQGQNQLYNELNLGRREEYDVLDKRRGR

SEQ ID NO: 24 - protein, 391 aa, synthetic construct
MALPVTALLLPLALLLHAARPSQFRVSPLDEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVVTITADESTSTAYMELSSLRSEDTAVYYCAREFVPVFLPAKPTTTPAPRPPTPAPTIASQPLSLRPEACRPAAGGAVHTRGLDFACDIYIWAPLAGTCGVLLLSLVITLYCNHRNQRRRFSVVKRGRKKLLYIFKQPFMRPVQTTQEEDGCSCRFPRVKFSRSADAPAYQQGQNQLYNELNLGRREEYDVLDKRRGRD

SEQ ID NO: 25 - protein, 392 aa, synthetic construct
MALPVTALLLPLALLLHAARPSQFRVSPLDEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVVTITADESTSTAYMELSSLRSEDTAVYYCAREFVPVFLPAKPTTTPAPRPPTPAPTIASQPLSLRPEACRPAAGGAVHTRGLDFACDIYIWAPLAGTCGVLLLSLVITLYCNHRNQRRRFSVVKRGRKKLLYIFKQPFMRPVQTTQEEDGCSCRFPRVKFSRSADAPAYQQGQNQLYNELNLGRREEYDVLDKRRGRDP

SEQ ID NO: 26 - protein, 393 aa, synthetic construct
MALPVTALLLPLALLLHAARPSQFRVSPLDEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVVTITADESTSTAYMELSSLRSEDTAVYYCAREFVPVFLPAKPTTTPAPRPPTPAPTIASQPLSLRPEACRPAAGGAVHTRGLDFACDIYIWAPLAGTCGVLLLSLVITLYCNHRNQRRRFSVVKRGRKKLLYIFKQPFMRPVQTTQEEDGCSCRFPRVKFSRSADAPAYQQGQNQLYNELNLGRREEYDVLDKRRGRDPE

SEQ ID NO: 27 - protein, 394 aa, synthetic construct
MALPVTALLLPLALLLHAARPSQFRVSPLDEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVVTITADESTSTAYMELSSLRSEDTAVYYCAREFVPVFLPAKPTTTPAPRPPTPAPTIASQPLSLRPEACRPAAGGAVHTRGLDFACDIYIWAPLAGTCGVLLLSLVITLYCNHRNQRRRFSVVKRGRKKLLYIFKQPFMRPVQTTQEEDGCSCRFPRVKFSRSADAPAYQQGQNQLYNELNLGRREEYDVLDKRRGRDPEM

SEQ ID NO: 28 - protein, 395 aa, synthetic construct
MALPVTALLLPLALLLHAARPSQFRVSPLDEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVVTITADESTSTAYMELSSLRSEDTAVYYCAREFVPVFLPAKPTTTPAPRPPTPAPTIASQPLSLRPEACRPAAGGAVHTRGLDFACDIYIWAPLAGTCGVLLLSLVITLYCNHRNQRRRFSVVKRGRKKLLYIFKQPFMRPVQTTQEEDGCSCRFPRVKFSRSADAPAYQQGQNQLYNELNLGRREEYDVLDKRRGRDPEMG

SEQ ID NO: 29 - protein, 396 aa, synthetic construct
MALPVTALLLPLALLLHAARPSQFRVSPLDEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVVTITADESTSTAYMELSSLRSEDTAVYYCAREFVPVFLPAKPTTTPAPRPPTPAPTIASQPLSLRPEACRPAAGGAVHTRGLDFACDIYIWAPLAGTCGVLLLSLVITLYCNHRNQRRRFSVVKRGRKKLLYIFKQPFMRPVQTTQEEDGCSCRFPRVKFSRSADAPAYQQGQNQLYNELNLGRREEYDVLDKRRGRDPEMGG

SEQ ID NO: 30 - protein, 397 aa, synthetic construct
MALPVTALLLPLALLLHAARPSQFRVSPLDEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVVTITADESTSTAYMELSSLRSEDTAVYYCAREFVPVFLPAKPTTTPAPRPPTPAPTIASQPLSLRPEACRPAAGGAVHTRGLDFACDIYIWAPLAGTCGVLLLSLVITLYCNHRNQRRRFSVVKRGRKKLLYIFKQPFMRPVQTTQEEDGCSCRFPRVKFSRSADAPAYQQGQNQLYNELNLGRREEYDVLDKRRGRDPEMGGK

SEQ ID NO: 31 - protein, 398 aa, synthetic construct
MALPVTALLLPLALLLHAARPSQFRVSPLDEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVVTITADESTSTAYMELSSLRSEDTAVYYCAREFVPVFLPAKPTTTPAPRPPTPAPTIASQPLSLRPEACRPAAGGAVHTRGLDFACDIYIWAPLAGTCGVLLLSLVITLYCNHRNQRRRFSVVKRGRKKLLYIFKQPFMRPVQTTQEEDGCSCRFPRVKFSRSADAPAYQQGQNQLYNELNLGRREEYDVLDKRRGRDPEMGGKP

SEQ ID NO: 32 - protein, 399 aa, synthetic construct
MALPVTALLLPLALLLHAARPSQFRVSPLDEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVVTITADESTSTAYMELSSLRSEDTAVYYCAREFVPVFLPAKPTTTPAPRPPTPAPTIASQPLSLRPEACRPAAGGAVHTRGLDFACDIYIWAPLAGTCGVLLLSLVITLYCNHRNQRRRFSVVKRGRKKLLYIFKQPFMRPVQTTQEEDGCSCRFPRVKFSRSADAPAYQQGQNQLYNELNLGRREEYDVLDKRRGRDPEMGGKPQ

SEQ ID NO: 33 - protein, 400 aa, synthetic construct
MALPVTALLLPLALLLHAARPSQFRVSPLDEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVVTITADESTSTAYMELSSLRSEDTAVYYCAREFVPVFLPAKPTTTPAPRPPTPAPTIASQPLSLRPEACRPAAGGAVHTRGLDFACDIYIWAPLAGTCGVLLLSLVITLYCNHRNQRRRFSVVKRGRKKLLYIFKQPFMRPVQTTQEEDGCSCRFPRVKFSRSADAPAYQQGQNQLYNELNLGRREEYDVLDKRRGRDPEMGGKPQR

SEQ ID NO: 34 - protein, 401 aa, synthetic construct
MALPVTALLLPLALLLHAARPSQFRVSPLDEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVVTITADESTSTAYMELSSLRSEDTAVYYCAREFVPVFLPAKPTTTPAPRPPTPAPTIASQPLSLRPEACRPAAGGAVHTRGLDFACDIYIWAPLAGTCGVLLLSLVITLYCNHRNQRRRFSVVKRGRKKLLYIFKQPFMRPVQTTQEEDGCSCRFPRVKFSRSADAPAYQQGQNQLYNELNLGRREEYDVLDKRRGRDPEMGGKPQRR

SEQ ID NO: 35 - protein, 381 aa, synthetic construct
MALPVTALLLPLALLLHAARPSQFRVSPLDEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVVTITADESTSTAYMELSSLRSEDTAVYYCAREFVPVFLPAKPTTTPAPRPPTPAPTIASQPLSLRPEACRPAAGGAVHTRGLDFACDIYIWAPLAGTCGVLLLSLVITLYCNHRNQRRKYRSNKGESPVEPAEPCRYSCPREEEGSTIPIQEDYRKPEPACSPRVKFSRSADAPAYQQGQNQLYNELN

SEQ ID NO: 36 - protein, 382 aa, synthetic construct
MALPVTALLLPLALLLHAARPSQFRVSPLDEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVVTITADESTSTAYMELSSLRSEDTAVYYCAREFVPVFLPAKPTTTPAPRPPTPAPTIASQPLSLRPEACRPAAGGAVHTRGLDFACDIYIWAPLAGTCGVLLLSLVITLYCNHRNQRRKYRSNKGESPVEPAEPCRYSCPREEEGSTIPIQEDYRKPEPACSPRVKFSRSADAPAYQQGQNQLYNELNL

SEQ ID NO: 37 - protein, 383 aa, synthetic construct
MALPVTALLLPLALLLHAARPSQFRVSPLDEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVVTITADESTSTAYMELSSLRSEDTAVYYCAREFVPVFLPAKPTTTPAPRPPTPAPTIASQPLSLRPEACRPAAGGAVHTRGLDFACDIYIWAPLAGTCGVLLLSLVITLYCNHRNQRRKYRSNKGESPVEPAEPCRYSCPREEEGSTIPIQEDYRKPEPACSPRVKFSRSADAPAYQQGQNQLYNELNLG

SEQ ID NO: 38 - protein, 384 aa, synthetic construct
MALPVTALLLPLALLLHAARPSQFRVSPLDEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVVTITADESTSTAYMELSSLRSEDTAVYYCAREFVPVFLPAKPTTTPAPRPPTPAPTIASQPLSLRPEACRPAAGGAVHTRGLDFACDIYIWAPLAGTCGVLLLSLVITLYCNHRNQRRKYRSNKGESPVEPAEPCRYSCPREEEGSTIPIQEDYRKPEPACSPRVKFSRSADAPAYQQGQNQLYNELNLGR

SEQ ID NO: 39 - protein, 385 aa, synthetic construct
MALPVTALLLPLALLLHAARPSQFRVSPLDEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVVTITADESTSTAYMELSSLRSEDTAVYYCAREFVPVFLPAKPTTTPAPRPPTPAPTIASQPLSLRPEACRPAAGGAVHTRGLDFACDIYIWAPLAGTCGVLLLSLVITLYCNHRNQRRKYRSNKGESPVEPAEPCRYSCPREEEGSTIPIQEDYRKPEPACSPRVKFSRSADAPAYQQGQNQLYNELNLGRR

SEQ ID NO: 40 - protein, 386 aa, synthetic construct
MALPVTALLLPLALLLHAARPSQFRVSPLDEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVVTITADESTSTAYMELSSLRSEDTAVYYCAREFVPVFLPAKPTTTPAPRPPTPAPTIASQPLSLRPEACRPAAGGAVHTRGLDFACDIYIWAPLAGTCGVLLLSLVITLYCNHRNQRRKYRSNKGESPVEPAEPCRYSCPREEEGSTIPIQEDYRKPEPACSPRVKFSRSADAPAYQQGQNQLYNELNLGRRE

SEQ ID NO: 41 - protein, 387 aa, synthetic construct
MALPVTALLLPLALLLHAARPSQFRVSPLDEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVVTITADESTSTAYMELSSLRSEDTAVYYCAREFVPVFLPAKPTTTPAPRPPTPAPTIASQPLSLRPEACRPAAGGAVHTRGLDFACDIYIWAPLAGTCGVLLLSLVITLYCNHRNQRRKYRSNKGESPVEPAEPCRYSCPREEEGSTIPIQEDYRKPEPACSPRVKFSRSADAPAYQQGQNQLYNELNLGRREE

SEQ ID NO: 42 - protein, 388 aa, synthetic construct
MALPVTALLLPLALLLHAARPSQFRVSPLDEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVVTITADESTSTAYMELSSLRSEDTAVYYCAREFVPVFLPAKPTTTPAPRPPTPAPTIASQPLSLRPEACRPAAGGAVHTRGLDFACDIYIWAPLAGTCGVLLLSLVITLYCNHRNQRRKYRSNKGESPVEPAEPCRYSCPREEEGSTIPIQEDYRKPEPACSPRVKFSRSADAPAYQQGQNQLYNELNLGRREEY

SEQ ID NO: 43 - protein, 389 aa, synthetic construct
MALPVTALLLPLALLLHAARPSQFRVSPLDEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVVTITADESTSTAYMELSSLRSEDTAVYYCAREFVPVFLPAKPTTTPAPRPPTPAPTIASQPLSLRPEACRPAAGGAVHTRGLDFACDIYIWAPLAGTCGVLLLSLVITLYCNHRNQRRKYRSNKGESPVEPAEPCRYSCPREEEGSTIPIQEDYRKPEPACSPRVKFSRSADAPAYQQGQNQLYNELNLGRREEYD

SEQ ID NO: 44 - protein, 390 aa, synthetic construct
MALPVTALLLPLALLLHAARPSQFRVSPLDEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVVTITADESTSTAYMELSSLRSEDTAVYYCAREFVPVFLPAKPTTTPAPRPPTPAPTIASQPLSLRPEACRPAAGGAVHTRGLDFACDIYIWAPLAGTCGVLLLSLVITLYCNHRNQRRKYRSNKGESPVEPAEPCRYSCPREEEGSTIPIQEDYRKPEPACSPRVKFSRSADAPAYQQGQNQLYNELNLGRREEYDV

SEQ ID NO: 45 - protein, 391 aa, synthetic construct
MALPVTALLLPLALLLHAARPSQFRVSPLDEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVVTITADESTSTAYMELSSLRSEDTAVYYCAREFVPVFLPAKPTTTPAPRPPTPAPTIASQPLSLRPEACRPAAGGAVHTRGLDFACDIYIWAPLAGTCGVLLLSLVITLYCNHRNQRRKYRSNKGESPVEPAEPCRYSCPREEEGSTIPIQEDYRKPEPACSPRVKFSRSADAPAYQQGQNQLYNELNLGRREEYDVL

SEQ ID NO: 46 - protein, 392 aa, synthetic construct
MALPVTALLLPLALLLHAARPSQFRVSPLDEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVVTITADESTSTAYMELSSLRSEDTAVYYCAREFVPVFLPAKPTTTPAPRPPTPAPTIASQPLSLRPEACRPAAGGAVHTRGLDFACDIYIWAPLAGTCGVLLLSLVITLYCNHRNQRRKYRSNKGESPVEPAEPCRYSCPREEEGSTIPIQEDYRKPEPACSPRVKFSRSADAPAYQQGQNQLYNELNLGRREEYDVLD

SEQ ID NO: 47 - protein, 393 aa, synthetic construct
MALPVTALLLPLALLLHAARPSQFRVSPLDEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVVTITADESTSTAYMELSSLRSEDTAVYYCAREFVPVFLPAKPTTTPAPRPPTPAPTIASQPLSLRPEACRPAAGGAVHTRGLDFACDIYIWAPLAGTCGVLLLSLVITLYCNHRNQRRKYRSNKGESPVEPAEPCRYSCPREEEGSTIPIQEDYRKPEPACSPRVKFSRSADAPAYQQGQNQLYNELNLGRREEYDVLDK

SEQ ID NO: 48 - protein, 394 aa, synthetic construct
MALPVTALLLPLALLLHAARPSQFRVSPLDEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVVTITADESTSTAYMELSSLRSEDTAVYYCAREFVPVFLPAKPTTTPAPRPPTPAPTIASQPLSLRPEACRPAAGGAVHTRGLDFACDIYIWAPLAGTCGVLLLSLVITLYCNHRNQRRKYRSNKGESPVEPAEPCRYSCPREEEGSTIPIQEDYRKPEPACSPRVKFSRSADAPAYQQGQNQLYNELNLGRREEYDVLDKR

SEQ ID NO: 49 - protein, 395 aa, synthetic construct
MALPVTALLLPLALLLHAARPSQFRVSPLDEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVVTITADESTSTAYMELSSLRSEDTAVYYCAREFVPVFLPAKPTTTPAPRPPTPAPTIASQPLSLRPEACRPAAGGAVHTRGLDFACDIYIWAPLAGTCGVLLLSLVITLYCNHRNQRRKYRSNKGESPVEPAEPCRYSCPREEEGSTIPIQEDYRKPEPACSPRVKFSRSADAPAYQQGQNQLYNELNLGRREEYDVLDKRR

SEQ ID NO: 50 - protein, 41 aa, synthetic construct
ERTSPTVPEKLGGGVGNSGARRWRGVNWESDVVYVVAVNVL

SEQ ID NO: 51 - protein, 42 aa, synthetic construct
ERTSPTVPEKLGGGVGNSGARRWRGVNWESDVVYVVAVNVLF

SEQ ID NO: 52 - protein, 43 aa, synthetic construct
ERTSPTVPEKLGGGVGNSGARRWRGVNWESDVVYVVAVNVLFR

SEQ ID NO: 53 - protein, 48 aa, synthetic construct
GARQWAERTSPTVPEGVGNSGARRWRGVNWVVAVNVLFRNGFAARTQV

SEQ ID NO: 54 - protein, 47 aa, synthetic construct
GARQWAERTSPTVPEGVGNSGARRWRGVNWVVAVNVLFRNGFAARTQ

SEQ ID NO: 55 - protein, 112 aa, synthetic construct
VKDPTCRFGKLGSRLGTERQQNMGQTGYLWAVPAPAQGQEQLEQQNMGQTGYLWAVPAPAQGQEQMVPRCGPALSSFRTIRCFQGAPRTNDPVPYLNPISSLLASVRALLLP

SEQ ID NO: 56 - protein, 113 aa, synthetic construct
VKDPTCRFGKLGSRLGTERQQNMGQTGYLWAVPAPAQGQEQLEQQNMGQTGYLWAVPAPAQGQEQMVPRCGPALSSFRTIRCFQGAPRTNDPVPYLNPISSLLASVRALLLPE

SEQ ID NO: 57 - protein, 114 aa, synthetic construct
VKDPTCRFGKLGSRLGTERQQNMGQTGYLWAVPAPAQGQEQLEQQNMGQTGYLWAVPAPAQGQEQMVPRCGPALSSFRTIRCFQGAPRTNDPVPYLNPISSLLASVRALLLPEL

SEQ ID NO: 58 - protein, 115 aa, synthetic construct
VKDPTCRFGKLGSRLGTERQQNMGQTGYLWAVPAPAQGQEQLEQQNMGQTGYLWAVPAPAQGQEQMVPRCGPALSSFRTIRCFQGAPRTNDPVPYLNPISSLLASVRALLLPELN

SEQ ID NO: 59 - protein, 115 aa, synthetic construct; identical to SEQ ID NO: 58

SEQ ID NO: 60 - protein, 116 aa, synthetic construct
VKDPTCRFGKLGSRLGTERQQNMGQTGYLWAVPAPAQGQEQLEQQNMGQTGYLWAVPAPAQGQEQMVPRCGPALSSFRTIRCFQGAPRTNDPVPYLNPISSLLASVRALLLPELNK

SEQ ID NO: 61 - DNA, 1131 bp, synthetic construct
  source 1..1131: mol_type = other DNA
atggcgctgccggtgaccgcgctgctgctgccgctggcgctgctgctgcatgcggcgcgcccgagccagtttcgcgtgagcccgctggatgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaatttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgccgctttagcgtggtgaaacgcggccgcaaaaaactgctgtatatttttaaacagccgtttatgcgcccggtgcagaccacccaggaagaagatggctgcagctgccgctttccgcgcgtgaaatttagccgcagcgcggatgcgccggcgtatcagcagggccagaaccagctgtataacgaactgaacctgggctga

SEQ ID NO: 62 - DNA, 1134 bp, synthetic construct
  source 1..1134: mol_type = other DNA
atggcgctgccggtgaccgcgctgctgctgccgctggcgctgctgctgcatgcggcgcgcccgagccagtttcgcgtgagcccgctggatgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaatttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgccgctttagcgtggtgaaacgcggccgcaaaaaactgctgtatatttttaaacagccgtttatgcgcccggtgcagaccacccaggaagaagatggctgcagctgccgctttccgcgcgtgaaatttagccgcagcgcggatgcgccggcgtatcagcagggccagaaccagctgtataacgaactgaacctgggccgctga

SEQ ID NO: 63 - DNA, 1137 bp, synthetic construct
  source 1..1137: mol_type = other DNA
atggcgctgccggtgaccgcgctgctgctgccgctggcgctgctgctgcatgcggcgcgcccgagccagtttcgcgtgagcccgctggatgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaatttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgccgctttagcgtggtgaaacgcggccgcaaaaaactgctgtatatttttaaacagccgtttatgcgcccggtgcagaccacccaggaagaagatggctgcagctgccgctttccgcgcgtgaaatttagccgcagcgcggatgcgccggcgtatcagcagggccagaaccagctgtataacgaactgaacctgggccgccgctga

SEQ ID NO: 64 - DNA, 1140 bp, synthetic construct
  source 1..1140: mol_type = other DNA
atggcgctgccggtgaccgcgctgctgctgccgctggcgctgctgctgcatgcggcgcgcccgagccagtttcgcgtgagcccgctggatgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaatttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgccgctttagcgtggtgaaacgcggccgcaaaaaactgctgtatatttttaaacagccgtttatgcgcccggtgcagaccacccaggaagaagatggctgcagctgccgctttccgcgcgtgaaatttagccgcagcgcggatgcgccggcgtatcagcagggccagaaccagctgtataacgaactgaacctgggccgccgcgaatga

SEQ ID NO: 65 - DNA, 1143 bp, synthetic construct
  source 1..1143: mol_type = other DNA
atggcgctgccggtgaccgcgctgctgctgccgctggcgctgctgctgcatgcggcgcgcccgagccagtttcgcgtgagcccgctggatgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaatttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgccgctttagcgtggtgaaacgcggccgcaaaaaactgctgtatatttttaaacagccgtttatgcgcccggtgcagaccacccaggaagaagatggctgcagctgccgctttccgcgcgtgaaatttagccgcagcgcggatgcgccggcgtatcagcagggccagaaccagctgtataacgaactgaacctgggccgccgcgaagaatga

SEQ ID NO: 66 - DNA, 1146 bp, synthetic construct
  source 1..1146: mol_type = other DNA
atggcgctgccggtgaccgcgctgctgctgccgctggcgctgctgctgcatgcggcgcgcccgagccagtttcgcgtgagcccgctggatgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaatttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgccgctttagcgtggtgaaacgcggccgcaaaaaactgctgtatatttttaaacagccgtttatgcgcccggtgcagaccacccaggaagaagatggctgcagctgccgctttccgcgcgtgaaatttagccgcagcgcggatgcgccggcgtatcagcagggccagaaccagctgtataacgaactgaacctgggccgccgcgaagaatattga

SEQ ID NO: 67 - DNA, 1149 bp, synthetic construct
  source 1..1149: mol_type = other DNA
atggcgctgccggtgaccgcgctgctgctgccgctggcgctgctgctgcatgcggcgcgcccgagccagtttcgcgtgagcccgctggatgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaatttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgccgctttagcgtggtgaaacgcggccgcaaaaaactgctgtatatttttaaacagccgtttatgcgcccggtgcagaccacccaggaagaagatggctgcagctgccgctttccgcgcgtgaaatttagccgcagcgcggatgcgccggcgtatcagcagggccagaaccagctgtataacgaactgaacctgggccgccgcgaagaatatgattga

SEQ ID NO: 68 - DNA, 1152 bp, synthetic construct
  source 1..1152: mol_type = other DNA
atggcgctgccggtgaccgcgctgctgctgccgctggcgctgctgctgcatgcggcgcgcccgagccagtttcgcgtgagcccgctggatgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaatttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgccgctttagcgtggtgaaacgcggccgcaaaaaactgctgtatatttttaaacagccgtttatgcgcccggtgcagaccacccaggaagaagatggctgcagctgccgctttccgcgcgtgaaatttagccgcagcgcggatgcgccggcgtatcagcagggccagaaccagctgtataacgaactgaacctgggccgccgcgaagaatatgatgtgtga

SEQ ID NO: 69 - DNA, 1155 bp, synthetic construct
  source 1..1155: mol_type = other DNA
atggcgctgccggtgaccgcgctgctgctgccgctggcgctgctgctgcatgcggcgcgcccgagccagtttcgcgtgagcccgctggatgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaatttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgccgctttagcgtggtgaaacgcggccgcaaaaaactgctgtatatttttaaacagccgtttatgcgcccggtgcagaccacccaggaagaagatggctgcagctgccgctttccgcgcgtgaaatttagccgcagcgcggatgcgccggcgtatcagcagggccagaaccagctgtataacgaactgaacctgggccgccgcgaagaatatgatgtgctgtga

SEQ ID NO: 70 - DNA, 1158 bp, synthetic construct
  source 1..1158: mol_type = other DNA
atggcgctgccggtgaccgcgctgctgctgccgctggcgctgctgctgcatgcggcgcgcccgagccagtttcgcgtgagcccgctggatgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaatttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgccgctttagcgtggtgaaacgcggccgcaaaaaactgctgtatatttttaaacagccgtttatgcgcccggtgcagaccacccaggaagaagatggctgcagctgccgctttccgcgcgtgaaatttagccgcagcgcggatgcgccggcgtatcagcagggccagaaccagctgtataacgaactgaacctgggccgccgcgaagaatatgatgtgctggattga

SEQ ID NO: 71 - DNA, 1161 bp, synthetic construct
  source 1..1161: mol_type = other DNA
atggcgctgccggtgaccgcgctgctgctgccgctggcgctgctgctgcatgcggcgcgcccgagccagtttcgcgtgagcccgctggatgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaatttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgccgctttagcgtggtgaaacgcggccgcaaaaaactgctgtatatttttaaacagccgtttatgcgcccggtgcagaccacccaggaagaagatggctgcagctgccgctttccgcgcgtgaaatttagccgcagcgcggatgcgccggcgtatcagcagggccagaaccagctgtataacgaactgaacctgggccgccgcgaagaatatgatgtgctggataaatga

SEQ ID NO: 72 - DNA, 1164 bp, synthetic construct
  source 1..1164: mol_type = other DNA
atggcgctgccggtgaccgcgctgctgctgccgctggcgctgctgctgcatgcggcgcgcccgagccagtttcgcgtgagcccgctggatgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaatttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgccgctttagcgtggtgaaacgcggccgcaaaaaactgctgtatatttttaaacagccgtttatgcgcccggtgcagaccacccaggaagaagatggctgcagctgccgctttccgcgcgtgaaatttagccgcagcgcggatgcgccggcgtatcagcagggccagaaccagctgtataacgaactgaacctgggccgccgcgaagaatatgatgtgctggataaacgctga

SEQ ID NO: 73 - DNA, 1167 bp, synthetic construct
  source 1..1167: mol_type = other DNA
atggcgctgccggtgaccgcgctgctgctgccgctggcgctgctgctgcatgcggcgcgcccgagccagtttcgcgtgagcccgctggatgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaatttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgccgctttagcgtggtgaaacgcggccgcaaaaaactgctgtatatttttaaacagccgtttatgcgcccggtgcagaccacccaggaagaagatggctgcagctgccgctttccgcgcgtgaaatttagccgcagcgcggatgcgccggcgtatcagcagggccagaaccagctgtataacgaactgaacctgggccgccgcgaagaatatgatgtgctggataaacgccgctga

SEQ ID NO: 74 - DNA, 1170 bp, synthetic construct
  source 1..1170: mol_type = other DNA
atggcgctgccggtgaccgcgctgctgctgccgctggcgctgctgctgcatgcggcgcgcccgagccagtttcgcgtgagcccgctggatgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaatttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgccgctttagcgtggtgaaacgcggccgcaaaaaactgctgtatatttttaaacagccgtttatgcgcccggtgcagaccacccaggaagaagatggctgcagctgccgctttccgcgcgtgaaatttagccgcagcgcggatgcgccggcgtatcagcagggccagaaccagctgtataacgaactgaacctgggccgccgcgaagaatatgatgtgctggataaacgccgcggctga

SEQ ID NO: 75 - DNA, 1173 bp, synthetic construct
  source 1..1173: mol_type = other DNA
atggcgctgccggtgaccgcgctgctgctgccgctggcgctgctgctgcatgcggcgcgcccgagccagtttcgcgtgagcccgctggatgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaatttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgccgctttagcgtggtgaaacgcggccgcaaaaaactgctgtatatttttaaacagccgtttatgcgcccggtgcagaccacccaggaagaagatggctgcagctgccgctttccgcgcgtgaaatttagccgcagcgcggatgcgccggcgtatcagcagggccagaaccagctgtataacgaactgaacctgggccgccgcgaagaatatgatgtgctggataaacgccgcggccgctga

SEQ ID NO: 76 - DNA, 1176 bp, synthetic construct
  source 1..1176: mol_type = other DNA
atggcgctgccggtgaccgcgctgctgctgccgctggcgctgctgctgcatgcggcgcgcccgagccagtttcgcgtgagcccgctggatgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaatttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgccgctttagcgtggtgaaacgcggccgcaaaaaactgctgtatatttttaaacagccgtttatgcgcccggtgcagaccacccaggaagaagatggctgcagctgccgctttccgcgcgtgaaatttagccgcagcgcggatgcgccggcgtatcagcagggccagaaccagctgtataacgaactgaacctgggccgccgcgaagaatatgatgtgctggataaacgccgcggccgcgattga

SEQ ID NO: 77 - DNA, 1179 bp, synthetic construct
  source 1..1179: mol_type = other DNA
atggcgctgccggtgaccgcgctgctgctgccgctggcgctgctgctgcatgcggcgcgcccgagccagtttcgcgtgagcccgctggatgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaatttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgccgctttagcgtggtgaaacgcggccgcaaaaaactgctgtatatttttaaacagccgtttatgcgcccggtgcagaccacccaggaagaagatggctgcagctgccgctttccgcgcgtgaaatttagccgcagcgcggatgcgccggcgtatcagcagggccagaaccagctgtataacgaactgaacctgggccgccgcgaagaatatgatgtgctggataaacgccgcggccgcgatccgtga

SEQ ID NO: 78 - DNA, 1182 bp, synthetic construct
  source 1..1182: mol_type = other DNA
atggcgctgccggtgaccgcgctgctgctgccgctggcgctgctgctgcatgcggcgcgcccgagccagtttcgcgtgagcccgctggatgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaatttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgccgctttagcgtggtgaaacgcggccgcaaaaaactgctgtatatttttaaacagccgtttatgcgcccggtgcagaccacccaggaagaagatggctgcagctgccgctttccgcgcgtgaaatttagccgcagcgcggatgcgccggcgtatcagcagggccagaaccagctgtataacgaactgaacctgggccgccgcgaagaatatgatgtgctggataaacgccgcggccgcgatccggaatga

SEQ ID NO: 79 - DNA, 1185 bp, synthetic construct
  source 1..1185: mol_type = other DNA
atggcgctgccggtgaccgcgctgctgctgccgctggcgctgctgctgcatgcggcgcgcccgagccagtttcgcgtgagcccgctggatgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaatttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgccgctttagcgtggtgaaacgcggccgcaaaaaactgctgtatatttttaaacagccgtttatgcgcccggtgcagaccacccaggaagaagatggctgcagctgccgctttccgcgcgtgaaatttagccgcagcgcggatgcgccggcgtatcagcagggccagaaccagctgtataacgaactgaacctgggccgccgcgaagaatatgatgtgctggataaacgccgcggccgcgatccggaaatgtga

SEQ ID NO: 80 - DNA, 1188 bp, synthetic construct
  source 1..1188: mol_type = other DNA
atggcgctgccggtgaccgcgctgctgctgccgctggcgctgctgctgcatgcggcgcgcccgagccagtttcgcgtgagcccgctggatgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaatttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgccgctttagcgtggtgaaacgcggccgcaaaaaactgctgtatatttttaaacagccgtttatgcgcccggtgcagaccacccaggaagaagatggctgcagctgccgctttccgcgcgtgaaatttagccgcagcgcggatgcgccggcgtatcagcagggccagaaccagctgtataacgaactgaacctgggccgccgcgaagaatatgatgtgctggataaacgccgcggccgcgatccggaaatgggctga

SEQ ID NO: 81 - DNA, 1191 bp, synthetic construct
  source 1..1191: mol_type = other DNA
atggcgctgccggtgaccgcgctgctgctgccgctggcgctgctgctgcatgcggcgcgcccgagccagtttcgcgtgagcccgctggatgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaatttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgccgctttagcgtggtgaaacgcggccgcaaaaaactgctgtatatttttaaacagccgtttatgcgcccggtgcagaccacccaggaagaagatggctgcagctgccgctttccgcgcgtgaaatttagccgcagcgcggatgcgccggcgtatcagcagggccagaaccagctgtataacgaactgaacctgggccgccgcgaagaatatgatgtgctggataaacgccgcggccgcgatccggaaatgggcggctga

SEQ ID NO: 82 - DNA, 1194 bp, synthetic construct
  source 1..1194: mol_type = other DNA
atggcgctgccggtgaccgcgctgctgctgccgctggcgctgctgctgcatgcggcgcgcccgagccagtttcgcgtgagcccgctggatgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaatttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgccgctttagcgtggtgaaacgcggccgcaaaaaactgctgtatatttttaaacagccgtttatgcgcccggtgcagaccacccaggaagaagatggctgcagctgccgctttccgcgcgtgaaatttagccgcagcgcggatgcgccggcgtatcagcagggccagaaccagctgtataacgaactgaacctgggccgccgcgaagaatatgatgtgctggataaacgccgcggccgcgatccggaaatgggcggcaaatga

SEQ ID NO: 83 - DNA, 1197 bp, synthetic construct
  source 1..1197: mol_type = other DNA
atggcgctgccggtgaccgcgctgctgctgccgctggcgctgctgctgcatgcggcgcgcccgagccagtttcgcgtgagcccgctggatgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaatttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgccgctttagcgtggtgaaacgcggccgcaaaaaactgctgtatatttttaaacagccgtttatgcgcccggtgcagaccacccaggaagaagatggctgcagctgccgctttccgcgcgtgaaatttagccgcagcgcggatgcgccggcgtatcagcagggccagaaccagctgtataacgaactgaacctgggccgccgcgaagaatatgatgtgctggataaacgccgcggccgcgatccggaaatgggcggcaaaccgtga

SEQ ID NO: 84 - DNA, 1200 bp, synthetic construct
  source 1..1200: mol_type = other DNA
atggcgctgccggtgaccgcgctgctgctgccgctggcgctgctgctgcatgcggcgcgcccgagccagtttcgcgtgagcccgctggatgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaatttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgccgctttagcgtggtgaaacgcggccgcaaaaaactgctgtatatttttaaacagccgtttatgcgcccggtgcagaccacccaggaagaagatggctgcagctgccgctttccgcgcgtgaaatttagccgcagcgcggatgcgccggcgtatcagcagggccagaaccagctgtataacgaactgaacctgggccgccgcgaagaatatgatgtgctggataaacgccgcggccgcgatccggaaatgggcggcaaaccgcagtga

SEQ ID NO: 85 - DNA, 1203 bp, synthetic construct
  source 1..1203: mol_type = other DNA
atggcgctgccggtgaccgcgctgctgctgccgctggcgctgctgctgcatgcggcgcgcccgagccagtttcgcgtgagcccgctggatgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaatttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgccgctttagcgtggtgaaacgcggccgcaaaaaactgctgtatatttttaaacagccgtttatgcgcccggtgcagaccacccaggaagaagatggctgcagctgccgctttccgcgcgtgaaatttagccgcagcgcggatgcgccggcgtatcagcagggccagaaccagctgtataacgaactgaacctgggccgccgcgaagaatatgatgtgctggataaacgccgcggccgcgatccggaaatgggcggcaaaccgcagcgctga

SEQ ID NO: 86 - DNA, 1206 bp, synthetic construct
  source 1..1206: mol_type = other DNA
atggcgctgccggtgaccgcgctgctgctgccgctggcgctgctgctgcatgcggcgcgcccgagccagtttcgcgtgagcccgctggatgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaatttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgccgctttagcgtggtgaaacgcggccgcaaaaaactgctgtatatttttaaacagccgtttatgcgcccggtgcagaccacccaggaagaagatggctgcagctgccgctttccgcgcgtgaaatttagccgcagcgcggatgcgccggcgtatcagcagggccagaaccagctgtataacgaactgaacctgggccgccgcgaagaatatgatgtgctggataaacgccgcggccgcgatccggaaatgggcggcaaaccgcagcgccgctga

SEQ ID NO: 87 - DNA, 1146 bp, synthetic construct
  source 1..1146: mol_type = other DNA
atggcgctgccggtgaccgcgctgctgctgccgctggcgctgctgctgcatgcggcgcgcccgagccagtttcgcgtgagcccgctggatgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaatttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgcaaatatcgcagcaacaaaggcgaaagcccggtggaaccggcggaaccgtgccgctatagctgcccgcgcgaagaagaaggcagcaccattccgattcaggaagattatcgcaaaccggaaccggcgtgcagcccgcgcgtgaaatttagccgcagcgcggatgcgccggcgtatcagcagggccagaaccagctgtataacgaactgaactga

SEQ ID NO: 88 - DNA, 1149 bp, synthetic construct
  source 1..1149: mol_type = other DNA
atggcgctgccggtgaccgcgctgctgctgccgctggcgctgctgctgcatgcggcgcgcccgagccagtttcgcgtgagcccgctggatgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaatttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgcaaatatcgcagcaacaaaggcgaaagcccggtggaaccggcggaaccgtgccgctatagctgcccgcgcgaagaagaaggcagcaccattccgattcaggaagattatcgcaaaccggaaccggcgtgcagcccgcgcgtgaaatttagccgcagcgcggatgcgccggcgtatcagcagggccagaaccagctgtataacgaactgaacctgtga

SEQ ID NO: 89 - DNA, 1152 bp, synthetic construct
  source 1..1152: mol_type = other DNA
atggcgctgccggtgaccgcgctgctgctgccgctggcgctgctgctgcatgcggcgcgcccgagccagtttcgcgtgagcccgctggatgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaatttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgcaaatatcgcagcaacaaaggcgaaagcccggtggaaccggcggaaccgtgccgctatagctgcccgcgcgaagaagaaggcagcaccattccgattcaggaagattatcgcaaaccggaaccggcgtgcagcccgcgcgtgaaatttagccgcagcgcggatgcgccggcgtatcagcagggccagaaccagctgtataacgaactgaacctgggctga

SEQ ID NO: 90 - DNA, 1155 bp, synthetic construct
  source 1..1155: mol_type = other DNA
atggcgctgccggtgaccgcgctgctgctgccgctggcgctgctgctgcatgcggcgcgcccgagccagtttcgcgtgagcccgctggatgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaatttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgcaaatatcgcagcaacaaaggcgaaagcccggtggaaccggcggaaccgtgccgctatagctgcccgcgcgaagaagaaggcagcaccattccgattcaggaagattatcgcaaaccggaaccggcgtgcagcccgcgcgtgaaatttagccgcagcgcggatgcgccggcgtatcagcagggccagaaccagctgtataacgaactgaacctgggccgctga

SEQ ID NO: 91 - DNA, 1158 bp, synthetic construct
  source 1..1158: mol_type = other DNA
atggcgctgccggtgaccgcgctgctgctgccgctggcgctgctgctgcatgcggcgcgcccgagccagtttcgcgtgagcccgctggatgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaatttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgcaaatatcgcagcaacaaaggcgaaagcccggtggaaccggcggaaccgtgccgctatagctgcccgcgcgaagaagaaggcagcaccattccgattcaggaagattatcgcaaaccggaaccggcgtgcagcccgcgcgtgaaatttagccgcagcgcggatgcgccggcgtatcagcagggccagaaccagctgtataacgaactgaacctgggccgccgctga

SEQ ID NO: 92 - DNA, 1161 bp, synthetic construct
  source 1..1161: mol_type = other DNA
atggcgctgccggtgaccgcgctgctgctgccgctggcgctgctgctgcatgcggcgcgcccgagccagtttcgcgtgagcccgctggatgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaatttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgcaaatatcgcagcaacaaaggcgaaagcccggtggaaccggcggaaccgtgccgctatagctgcccgcgcgaagaagaaggcagcaccattccgattcaggaagattatcgcaaaccggaaccggcgtgcagcccgcgcgtgaaatttagccgcagcgcggatgcgccggcgtatcagcagggccagaaccagctgtataacgaactgaacctgggccgccgcgaatga

SEQ ID NO: 93 - DNA, 1164 bp, synthetic construct
  source 1..1164: mol_type = other DNA
atggcgctgccggtgaccgcgctgctgctgccgctggcgctgctgctgcatgcggcgcgcccgagccagtttcgcgtgagcccgctggatgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaatttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgcaaatatcgcagcaacaaaggcgaaagcccggtggaaccggcggaaccgtgccgctatagctgcccgcgcgaagaagaaggcagcaccattccgattcaggaagattatcgcaaaccggaaccggcgtgcagcccgcgcgtgaaatttagccgcagcgcggatgcgccggcgtatcagcagggccagaaccagctgtataacgaactgaacctgggccgccgcgaagaatga

SEQ ID NO: 94 - DNA, 1167 bp, synthetic construct
  source 1..1167: mol_type = other DNA
atggcgctgccggtgaccgcgctgctgctgccgctggcgctgctgctgcatgcggcgcgcccgagccagtttcgcgtgagcccgctggatgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaatttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgcaaatatcgcagcaacaaaggcgaaagcccggtggaaccggcggaaccgtgccgctatagctgcccgcgcgaagaagaaggcagcaccattccgattcaggaagattatcgcaaaccggaaccggcgtgcagcccgcgcgtgaaatttagccgcagcgcggatgcgccggcgtatcagcagggccagaaccagctgtataacgaactgaacctgggccgccgcgaagaatattga

SEQ ID NO: 95 - DNA, 1170 bp, synthetic construct
  source 1..1170: mol_type = other DNA
atggcgctgccggtgaccgcgctgctgctgccgctggcgctgctgctgcatgcggcgcgcccgagccagtttcgcgtgagcccgctggatgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaatttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgcaaatatcgcagcaacaaaggcgaaagcccggtggaaccggcggaaccgtgccgctatagctgcccgcgcgaagaagaaggcagcaccattccgattcaggaagattatcgcaaaccggaaccggcgtgcagcccgcgcgtgaaatttagccgcagcgcggatgcgccggcgtatcagcagggccagaaccagctgtataacgaactgaacctgggccgccgcgaagaatatgattga

SEQ ID NO: 96 - DNA, 1173 bp, synthetic construct
  source 1..1173: mol_type = other DNA
atggcgctgccggtgaccgcgctgctgctgccgctggcgctgctgctgcatgcggcgcgcccgagccagtttcgcgtgagcccgctggatgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaatttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgcaaatatcgcagcaacaaaggcgaaagcccggtggaaccggcggaaccgtgccgctatagctgcccgcgcgaagaagaaggcagcaccattccgattcaggaagattatcgcaaaccggaaccggcgtgcagcccgcgcgtgaaatttagccgcagcgcggatgcgccggcgtatcagcagggccagaaccagctgtataacgaactgaacctgggccgccgcgaagaatatgatgtgtga

SEQ ID NO: 97 - DNA, 1176 bp, synthetic construct
  source 1..1176: mol_type = other DNA
atggcgctgccggtgaccgcgctgctgctgccgctggcgctgctgctgcatgcggcgcgcccgagccagtttcgcgtgagcccgctggatgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaatttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgcaaatatcgcagcaacaaaggcgaaagcccggtggaaccggcggaaccgtgccgctatagctgcccgcgcgaagaagaaggcagcaccattccgattcaggaagattatcgcaaaccggaaccggcgtgcagcccgcgcgtgaaatttagccgcagcgcggatgcgccggcgtatcagcagggccagaaccagctgtataacgaactgaacctgggccgccgcgaagaatatgatgtgctgtga

SEQ ID NO: 98 - DNA, 1179 bp, synthetic construct
  source 1..1179: mol_type = other DNA
atggcgctgccggtgaccgcgctgctgctgccgctggcgctgctgctgcatgcggcgcgcccgagccagtttcgcgtgagcccgctggatgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaatttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgcaaatatcgcagcaacaaaggcgaaagcccggtggaaccggcggaaccgtgccgctatagctgcccgcgcgaagaagaaggcagcaccattccgattcaggaagattatcgcaaaccggaaccggcgtgcagcccgcgcgtgaaatttagccgcagcgcggatgcgccggcgtatcagcagggccagaaccagctgtataacgaactgaacctgggccgccgcgaagaatatgatgtgctggattga

SEQ ID NO: 99 - DNA, 1182 bp, synthetic construct
  source 1..1182: mol_type = other DNA
atggcgctgccggtgaccgcgctgctgctgccgctggcgctgctgctgcatgcggcgcgcccgagccagtttcgcgtgagcccgctggatgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaatttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgcaaatatcgcagcaacaaaggcgaaagcccggtggaaccggcggaaccgtgccgctatagctgcccgcgcgaagaagaaggcagcaccattccgattcaggaagattatcgcaaaccggaaccggcgtgcagcccgcgcgtgaaatttagccgcagcgcggatgcgccggcgtatcagcagggccagaaccagctgtataacgaactgaacctgggccgccgcgaagaatatgatgtgctggataaatga

SEQ ID NO: 100 - DNA, 1185 bp, synthetic construct
  source 1..1185: mol_type = other DNA
atggcgctgccggtgaccgcgctgctgctgccgctggcgctgctgctgcatgcggcgcgcccgagccagtttcgcgtgagcccgctggatgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaatttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgcaaatatcgcagcaacaaaggcgaaagcccggtggaaccggcggaaccgtgccgctatagctgcccgcgcgaagaagaaggcagcaccattccgattcaggaagattatcgcaaaccggaaccggcgtgcagcccgcgcgtgaaatttagccgcagcgcggatgcgccggcgtatcagcagggccagaaccagctgtataacgaactgaacctgggccgccgcgaagaatatgatgtgctggataaacgctga

SEQ ID NO: 101 - DNA, 1188 bp, synthetic construct
  source 1..1188: mol_type = other DNA
atggcgctgccggtgaccgcgctgctgctgccgctggcgctgctgctgcatgcggcgcgcccgagccagtttcgcgtgagcccgctggatgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaatttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgcaaatatcgcagcaacaaaggcgaaagcccggtggaaccggcggaaccgtgccgctatagctgcccgcgcgaagaagaaggcagcaccattccgattcaggaagattatcgcaaaccggaaccggcgtgcagcccgcgcgtgaaatttagccgcagcgcggatgcgccggcgtatcagcagggccagaaccagctgtataacgaactgaacctgggccgccgcgaagaatatgatgtgctggataaacgccgctga

SEQ ID NO: 102 - DNA, 585 bp, synthetic construct
  source 1..585: mol_type = other DNA
gaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtggtgaccattaccgcggatgaaagcaccagcaccgcgtatatggaactgagcagcctgcgcagcgaagataccgcggtgtattattgcgcgcgcgaa

SEQ ID NO: 103 - DNA, 489 bp, synthetic construct
  source 1..489: mol_type = other DNA
gaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagccgctttacctttggcccgggcaccaaagtggatattaaaggcagcaccagcggcagcggcaaaccgggcagcggcgaaggcagcaccaaaggccaggtgcagctggtgcagagcggcgcggaagtgaaaaaaccgggcagcagcgtgaaagtgagctgcaaagatagcggcggcacctttagcagctatgcgattagctgggtg

SEQ ID NO: 104 - DNA, 489 bp, synthetic construct; identical to SEQ ID NO: 103
  source 1..489: mol_type = other DNA

SEQ ID NO: 105 - DNA, 93 bp, synthetic construct
  source 1..93: mol_type = other DNA
atggcgctgccggtgaccgcgctgctgctgccgctggcgctgctgctgcatgcggcgcgcccgagccagtttcgcgtgagcccgctggattga

SEQ ID NO: 106 - DNA, 513 bp, synthetic construct
  source 1..513: mol_type = other DNA
tttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccagcgccgcaaatatcgcagcaacaaaggcgaaagcccggtggaaccggcggaaccgtgccgctatagctgcccgcgcgaagaagaaggcagcaccattccgattcaggaagattatcgcaaaccggaaccggcgtgcagcccgcgcgtgaaatttagccgcagcgcggatgcgccggcgtatcagcagggccagaaccagctgtataacgaactgaacctgggccgccgcgaagaatatgatgtgctggataaacgccgcggc

SEQ ID NO: 107 - DNA, 1143 bp, synthetic construct; identical to SEQ ID NO: 65
  source 1..1143: mol_type = other DNA

SEQ ID NO: 108 - DNA, 513 bp, synthetic construct; identical to SEQ ID NO: 106
  source 1..513: mol_type = other DNA

SEQ ID NO: 109 - DNA, 513 bp, synthetic construct; identical to SEQ ID NO: 106
  source 1..513: mol_type = other DNA